(19) United States
(12) Patent Application Publication
(10) Pub. No.: US 2015/0197766 A1
(43) Pub. Date: Jul. 16, 2015

(54) METHODS AND COMPOSITIONS FOR PRODUCTION OF RECOMBINANT PHARMACEUTICAL PROTEINS IN MEDICINAL PLANTS

(71) Applicant: PHARMA GREEN LLC, Feasterville-Trevose, PA (US)

(72) Inventors: Natalia POGREBNYAK, Highland Park, NJ (US); Vyacheslav ANDRIANOV, Warrington, PA (US); Maxim GOLOVKIN, Huntingdon Valley, PA (US)

(73) Assignee: PHARMA GREEN LLC, Feasterville-Trevose, PA (US)

(21) Appl. No.: 14/673,914

(22) Filed: Mar. 31, 2015

Related U.S. Application Data
(63) Continuation of application No. PCT/US2013/063086, filed on Oct. 2, 2013.
Application No. PCT/US2013/063086 is a continuation-in-part of application No. 13/849,154, filed on Mar. 22, 2013.
Application No. 13/849,154 is a continuation-in-part of application No. 13/922,719, filed on Jun. 20, 2013.
(60) Provisional application No. 61/709,186, filed on Oct. 3, 2012.
(60) Provisional application No. 61/614,167, filed on Mar. 22, 2012.
(60) Provisional application No. 61/663,271, filed on Jun. 22, 2012.

(51) Int. Cl. C12N 15/82; A61K 38/16; C07K 16/12; A01H 1/06; A61K 39/285

(52) U.S. Cl. CPC C12N 15/8258; C12N 15/8257; A01H 1/06; A61K 39/285; C07K 16/1278; A61K 38/168; C07K 2319/00; A61K 2039/58

(57) ABSTRACT

Methods of tissue culture and in vitro propagation of medicinal plants, in particular, plants of the genera Hydrastis, Echinacea, Kalanhoe, Thymus and Calendula are described. Methods of genetically engineering the medicinal plants are also described, along with methods of producing recombinant proteins in such plants. Compositions and methods for administering recombinant proteins produced in these plants to subjects in need thereof are provided.

### CROSS REFERENCE TO RELATED APPLICATIONS

This application is a continuation of International patent application No. PCT/US2013/063086, filed Oct. 2, 2013, which claims the benefit of U.S. provisional application No. 61/709,186, filed Oct. 3, 2012, U.S. patent application Ser. No. 13/849,154, filed Mar. 22, 2013, and U.S. patent application Ser. No. 13/922,719, filed Jun. 20, 2013. This application is also a continuation-in-part of U.S. patent application Ser. No. 13/849,154, filed Mar. 22, 2013, which claims the benefit of U.S. provisional application No. 61/614,167, filed Mar. 22, 2012. This application is also a continuation-in-part of U.S. patent application Ser. No. 13/922,719, filed Jun. 20, 2013, which claims the benefit of U.S. provisional application No. 61/663,271, filed Jun. 22, 2012, all of which are incorporated herein by reference as if fully set forth.

The sequence listing electronically filed with this application titled “Sequence Listing,” which was created on Mar. 31, 2015 and had a size of 7,621 bytes is incorporated by reference herein as if fully set forth.

### FIELD OF THE INVENTION

The present disclosure relates to methods and compositions to produce recombinant proteins in medicinal plants. In particular, the invention provides examples of genetically engineered plants of the genus Hydrastis, Echinacea, Thymus, Calendula or Kalanchoe comprising recombinant proteins, methods for producing recombinant proteins in such plants and corresponding plant-derived compositions. Methods of administering plant-derived compositions to subjects in need thereof are also described.

### BACKGROUND

Plants can be utilized as a biotechnology platform for industrial production of recombinant proteins. The advantages of plants compared to other production systems, e.g., bacteria, yeast, insect and mammalian cells, are lower production costs and the potential for easy scaling up production of large quantities of biomass. An additional advantage of plant-derived compositions is that these are free of endotoxins or human pathogens.

Efforts to produce commercial pharmaceutical proteins in plants are mainly focused on utilizing model plant species that are easy to transform, e.g., tobacco and Arabidopsis, and crop species that can be used for food or animal feed, e.g., tomato, alfalfa, lettuce, carrot, potato, cauliflower, maize and rice. See Golovkin, 2011, Production of Recombinant Pharmaceuticals Using Plant Biotechnology, In: Bioprocess Science and Technology, Series Biochemistry Research Trends Ed. Min-Tze Liong. Nova Sci. Publ., Inc., USA. ISBN: 978-1-61122-950; Gruskin, 2012 Nat Biotech 30: 211; and Pogrebnyak et al. 2006 Plant Sci 171: 677.

### SUMMARY

An aspect of the invention relates to a genetically engineered plant. The plant belongs to the genus selected from the group consisting of: Hydrastis, Echinacea, Thymus, Calendula and Kalanchoe.

An aspect of the invention relates to a method for genetically engineering a plant. The method includes contacting a plant with a vector comprising a nucleic acid encoding a recombinant protein. The method also includes selecting a genetically engineered plant expressing the recombinant protein. The plant belongs to the genus selected from the group consisting of: Hydrastis, Echinacea, Thymus, Calendula and Kalanchoe.

An aspect of the invention relates to a method for genetically engineering a plant. The method includes obtaining a mutant plant. The plant belongs to the genus selected from the group consisting of: Hydrastis, Echinacea, Thymus, Calendula and Kalanchoe.

An aspect of the invention relates to a method for producing a recombinant protein in a plant. The method includes genetically engineering the plant to includes a nucleic acid encoding the recombinant protein. The method also includes culturing a genetically engineered plant under conditions effective for expression a recombinant protein. The plant belongs to the genus selected from the group consisting of: Hydrastis, Echinacea, Thymus, Calendula and Kalanchoe.

An aspect of the invention relates to a method of treating a subject against a disease. The method includes genetically engineering a plant to include a nucleic acid encoding a recombinant protein capable of preventing, curing or eliminating at least one symptom of the disease in the subject. The plant belongs to the genus selected from the group consisting of: Hydrastis, Echinacea, Thymus, Calendula and Kalanchoe. The method includes harvesting a genetically engineered plant expressing the recombinant protein. The method includes performing the step (i) or (ii). The step (i) includes preparing a first composition that includes the genetically engineered plant, or a part thereof. The step (ii) includes isolating the recombinant protein and preparing a second composition that includes the isolated recombinant protein. The method also includes administering the first composition or the second composition to the subject in need thereof.

An aspect of the invention relates to a method of propagating a plant in vitro. The method includes culturing a plant, or a part of the plant, on a culture medium that includes at least one plant growth regulator. The method includes recovering multiple shoots from the plant or the part of the plant. The plant belongs to the genus selected from the group consisting of: Hydrastis, Echinacea, Thymus, Calendula and Kalanchoe.

An aspect of the invention relates a method of producing a cell suspension culture. The method includes culturing a plant, part, or tissue thereof, in a liquid culture medium that includes at least one auxin. The plant belongs to the genus selected from the group consisting of: Hydrastis, Echinacea, Thymus, Calendula and Kalanchoe.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIGS. 1A-1C illustrate development of in vitro propagation of Goldenseal (Hydrastis canadensis) under sterile conditions and climate-controlled environment. FIG. 1A illustrates multiple shoots induced from the leaf explants after six weeks of culturing on MSR1 medium with additional three weeks on MSR4. FIG. 1B illustrates shoot development from leaf tissues after 7 weeks of culturing on MSR3 medium. FIG. 1C illustrates elongation of shoots developed from the leaf explants after transferring them to MSR4 medium.

FIG. 2 illustrates in vitro propagation of Thyme (Thymus vulgaris) under sterile conditions and climate-controlled environment.

FIG. 3 illustrates in vitro mass propagation of Echinacea (Echinacea purpurea) with multiple shoot regeneration events from leaf explants on MSR5 medium.

FIG. 4 illustrates in vitro development of multiple shoots from Kalanchoe (Kalanchoe pinnata) leaf explants on MSR6 medium.

FIG. 5 illustrates shoot regeneration from Calendula (Calendula officinalis) cotyledons on MSR3 medium.

FIGS. 6A-6D illustrate in vitro initiation and propagation of callus and cell suspension. FIG. 6A illustrates callus induction from leaf tissues after 6 weeks culturing on MSC1 medium. FIG. 6B illustrates callus growth after 8 weeks culturing on MSC2 medium. FIG. 6C illustrates cell suspension after 2 weeks of culturing in liquid MS medium supplemented with 1 mg/l 2,4-D. FIG. 6D illustrates shoot regeneration from callus tissues after 8 weeks culturing on MSR3 medium.

FIGS. 7A-7B illustrate formation of the transgenic Goldenseal shoots on the selection medium after Agrobacterium-mediated transformation. FIG. 7A illustrates transgenic shoots developed on MST4 medium in the presence of kanamycin. FIG. 7B illustrates the magnified shoots from FIG. 7A.

FIG. 8 illustrates the morphologically normal transgenic Goldenseal plant transplanted into soil.

FIG. 9 illustrates histochemical GUS analysis of transgenic Calendula plants.

FIGS. 10A-10C illustrate schematic drawings of binary pBI-based vectors prepared for stable transformation of plants via Agrobacterium tumefaciens. FIG. 10A illustrates a vector for production of a TBL-Fc protein ATR-Fc. FIG. 10B illustrates a vector for cyanovirin (CNVR) microbicide generation. FIG. 10C illustrates a vector designed for production of the scytovirin (SCTV) microbicide.

FIGS. 11A-11D illustrate generation and analysis of the transgenic Echinacea plants. FIG. 11A illustrates the putative transgenic shoots. FIG. 11B illustrates target gene-specific PCR analysis of putative transgenic Echinacea plants selected on Km selective media. FIG. 11C illustrates Western blot detection of the recombinant protein target by a polyclonal mouse primary antibody against the E. coli-produced microbicide protein. FIG. 11D illustrates detection of the recombinant CNVR protein using ELISA.

FIGS. 12A-12B illustrate generation and analysis of the transgenic Kalanchoe plants. FIG. 12A illustrates the stringency of selection. FIG. 12B illustrates a target-specific detection of recombinant protein TBL in the extracts from the transgenic Kalanchoe plant lines using ELISA.

### DETAILED DESCRIPTION OF THE PREFERRED EMBODIMENTS

Certain terminology is used in the following description for convenience only and is not limiting. The words “right,” “left,” “top,” and “bottom” designate directions in the drawings to which reference is made.

An embodiment herein provides a genetically engineered plant. The genetically engineered plant may be a medicinal plant. The genetically engineered plant may belong to but is not limited to genus Hydrastis, Echinacea, Thymus, Calendula or Kalanchoe. The genetically engineered plant may be Hydrastis canadensis. The genetically engineered plant may be Echinacea purpurea. The genetically engineered plant may be Thymus vulgaris. The genetically engineered plant may be Calendula officinalis. The genetically engineered plant may be Kalanchoe pinnata.

In an embodiment, the plant may be genetically engineered to include a nucleic acid encoding a recombinant protein. The nucleic acid may be an exogenous nucleic acid. The exogenous nucleic acid may include genetic material not found in a native medicinal plant. The exogenous nucleic acid may include multiple exogenous nucleic acids. Multiple exogenous nucleic acids may originate from multiple sources or organisms. The nucleic acid may be generated recombinantly or synthetically, with a series of specified nucleic acid elements, which permit transcription of a particular nucleic acid in plant cells and plant tissues. The nucleic acid may be incorporated into a plasmid, chromosome, mitochondrial DNA, plastid DNA, virus or nucleic acid fragment. The nucleic acid may include an open reading frame encoding a recombinant protein.

In an embodiment, the recombinant protein may be a pharmaceutical protein. The pharmaceutical protein may be any protein to treat diseases. The pharmaceutical protein may be a microbicide. The term “microbicide” refers to any compound or substance capable of reducing the infectivity of microbes. Microbicides may reduce infectivity of viruses or bacteria. For example, microbicides may be applied inside the vagina or rectum to protect against sexually transmitted infections including human immunodeficiency virus (HIV). They may be formulated as gels, creams, films, or suppositories, used for preventing transmission of HIV. The microbicide may be griffithsin produced by red algae. See Mori et al. 2005 J Biol Chem 280: 9345, which is incorporated by reference herein as if fully set forth. The microbicide may be a cyanovirin as described by Huskens and Schols. See Huskens and Schols, 2012, Algal Lectins as Potential HIV Microbicide Candidates, Marine Drugs, 10, 1476-1497, which is incorporated herein by reference as if fully set forth. The amino acid sequence of the cyanovirin may be optimized for targeting apoplast. The cyanovirin may include, consist essentially of, or consist of a sequence with at least 70, 72, 75, 80, 85, 90, 91, 92, 93, 94, 95, 96, 97, 98, 99, or 100% identity to a reference sequence of SEQ ID NO: 1

The microbicide may be scytovirin. The amino acid sequence of the scytovirin may be optimized for targeting apoplast. The scytovirin may include, consist essentially of, or consist of a sequence with at least 70, 72, 75, 80, 85, 90, 91, 92, 93, 94, 95, 96, 97, 98, 99, or 100% identity to a reference sequence of SEQ ID NO: 3.

In an embodiment, the recombinant protein may be an antibody effective for eliminating or reducing the size of tumors in a subject having cancer. The antibody may have an ability to recognize and specifically bind to a target molecule associated with cells, tissues or organs affected by a disease. The target molecule may be a protein, a polypeptide, a peptide, a carbohydrate, a polynucleotide, a lipid, or combinations of at least two of the foregoing through at least one antigen recognition site within the variable region of the antibody. The antibody may specifically bind to a cancer stem cell marker protein and interfere with, for example, ligand binding, receptor dimerization, expression of a cancer stem cell marker protein, and/or downstream signaling of a cancer stem cell marker protein.

The antibody may be an anthrax toxin binding recombinant antibody. The anthrax toxin binding recombinant antibody may include a toxin binding ligand. The toxin binding ligand may be capable of binding anthrax toxin with high affinity. The toxin binding ligand may be a human or animal anthrax receptor (ATR) protein. The toxin binding ligand may be a capillary morphogenesis protein 2 (CMG-2). The toxin binding ligand may be a soluble domain of the CMG-2. The toxin binding ligand may be a soluble domain of another ATR protein. The toxin binding ligand may be a soluble domain of another anthrax toxin-binding polypeptide.

The toxin binding ligand may be a polypeptide capable of high affinity binding to a protective antigen (PA) region necessary for PA interaction with a lethal factor (LF) or an edema factor (EF) components of the anthrax toxin. The toxin binding ligand may be a protective antigen binding domain of a lethal factor (PA-LF; component A2).

The antibody may be a polyclonal antibody, an intact monoclonal antibody, an antibody fragment or fusion, which may be, but is not limited to, Fab, Fab′, F(ab′)2, an Fv fragment, a single chain Fv (scFv) mutant, a chimeric antibody or a multi-specific antibody. A multi-specific antibody may be a bi-specific antibody generated from at least two intact antibodies. The antibody may be a humanized antibody or a human antibody. The antibody may be a fusion protein comprising an antigen determination portion of an antibody. The antibody may be a fusion chimeric antibody against anthrax toxin consisting of anthrax Toxin Binding Ligand (TBL) polypeptide fused with Fc fragment of IgG, IgA or IgM antibody.

In an embodiment, the recombinant protein may be an antigen. The term “antigen” refers to a molecule that is capable of stimulating a recipient's immune system to produce an antigen-specific response, i.e., an immune response. Such an immune response may be a cellular immune response to an antigenic site present and/or a humoral immune response. The antigens may be virus coat proteins or membrane proteins. The viral coat proteins may include but are not limited to L1, B5 and A33 Vaccinia virus proteins. The antigen may be a Vaccinia virus glycoprotein B5 membrane antigen. The antigens may be capable of eliciting an immune response against poxvirus. The antigens may be capable of eliciting an immune response against Vaccinia virus.

In an embodiment, the genetically engineered plant may include a nucleic acid sequence optimized for protein expression in plants. The optimized sequence may enhance expression of an exogenous polynucleotide in plants. The optimized nucleic acid sequence may include plant optimized codon sequences. The nucleic acid may include, consist essentially, or consist of a sequence with at least 70, 72, 75, 80, 85, 90, 91, 92, 93, 94, 95, 96, 97, 98, 99, or 100% identity to a reference sequence selected from the group consisting of: SEQ ID NO: 2, SEQ ID NO: 4, SEQ ID NO: 5, and SEQ ID NO: 6.

Determining percent identity of two amino acid sequences or two nucleic acid sequences may include aligning and comparing the amino acid residues or nucleotides at corresponding positions in the two sequences. If all positions in two sequences are occupied by identical amino acid residues or nucleotides then the sequences are said to be 100% identical. Percent identity may be measured by the Basic Local Alignment Search Tool (BLAST; Altschul, S. F., Gish, W., Miller, W., Myers, E. W. & Lipman, D. J, 1990 “Basic local alignment search tool.” J. Mol. Biol. 215:403-410, which is incorporated herein by reference as if fully set forth).

In an embodiment, the recombinant protein may be a variant. Variants may include conservative amino acid substitutions, i.e., substitutions with amino acids having similar properties. Conservative substitutions may be a polar for polar amino acid (Glycine (G), Serine (S), Threonine (T), Tyrosine (Y), Cysteine (C), Asparagine (N) and Glutamine (Q)); a non-polar for non-polar amino acid (Alanine (A), Valine (V), Thyptophan (W), Leucine (L), Proline (P), Methionine (M), Phenilalanine (F)); acidic for acidic amino acid Aspartic acid (D), Glutamic acid (E)); basic for basic amino acid (Arginine (R), Histidine (H), Lysine (K)); charged for charged amino acids (Aspartic acid (D), Glutamic acid (E), Histidine (H), Lysine (K) and Arginine (R)); and a hydrophobic for hydrophobic amino acid (Alanine (A), Leucine (L), Isoleucine (I), Valine (V), Proline (P), Phenilalanine (F), Tryptophan (W) and Methionone (M)). Conservative nucleotide substitutions may be made in a nucleic acid sequence by substituting a codon for an amino acid with a different codon for the same amino acid. Variants may include non-conservative substitutions.

The recombinant protein may include a full length protein or a fragment. The fragment of the recombinant protein refers to a subsequence of the polypeptides herein that retain the biological function of the full length protein.

In an embodiment, fragments of a cyanovirin, a scytovirin, an anthrax toxin binding recombinant antibody or a Vaccinia virus glycoprotein B5 membrane antigen are provided. Fragments may include 50, 100, 150, 200, 300, 400, 600, or 700 contiguous amino acids or more.

The functionality of a recombinant protein, variants or fragments thereof, may be determined using any methods. The functionality may include conferring ability to bind the components of the anthrax toxin in a solution as determined by immunodetection methods. The functionality may be assessed in vitro using biochemical assays or live cells. The functionality of a protein, or variants, or fragments thereof, may be assessed based on the ability to protect subjects following of the infection of subjects with the causative agent. The functionality of a protein, or variants, or fragments thereof, may be assessed based on the ability to protect subjects after administering of a recombinant antitoxin following of the infection of animals with the causative agent of anthrax. The functionality may be assessed based on the ability to inhibit poxvirus. Assessment of functionality of proteins may include a virus neutralization assay which includes incubation of a virus titer with serial dilutions of serum obtained from an animal after periodic administering of an immunogenic protein and quantifying the amount of the remaining virus by, e.g., plaque “comet inhibition” assay (Isaacs et al., 1992 J Virol 66:7217; Aldaz-Carroll et al., 2005 J Virol. 79:6260; Xiao et al., 2006 Vaccine 25:1214, all of which are incorporated by reference herein as if fully set forth).

In an embodiment, a method for genetically engineering a plant is provided. The method may include contacting a plant with a vector. The vector may include a nucleic acid encoding a recombinant protein. The method may include selecting a genetically engineered plant expressing the recombinant protein.

In an embodiment, the plant may be genetically engineered using transformation. For transformation, the nucleic acid may be introduced into a genetic vector. Suitable vectors may be cloning vectors, transformation vectors, expression vectors, or virus-based vectors. The expression cassette portion of a vector may further include a regulatory element operably linked to at least one of the first polynucleotide, the second polynucleotide or the third polynucleotide. In this context, operably linked means that the regulatory element imparts its function on the nucleic acid. For example, a regulatory element may be a promoter, and the operably linked promoter would control overexpression of the nucleic acid.

The expression of the nucleic acid of the expression cassette may be under the control of a promoter which provides for transcription of the nucleic acid in a plant. The promoter may be a constitutive promoter or, tissue specific, or an inducible promoter. A constitutive promoter may provide transcription of the nucleic acid throughout most cells and tissues of the plant and during many stages of development but not necessarily all stages. An inducible promoter may initiate transcription of the nucleic acid sequence only when exposed to a particular chemical or environmental stimulus. A tissue specific promoter may be capable of initiating transcription in a particular plant tissue. Plant tissue may be, but is not limited to, a stem, leaves, trichomes, anthers, or seed. Constitutive promoter may be, but is not limited to, the Cauliflower Mosaic Virus (CAMV) 35S promoter, the Cestrum Yellow Leaf Curling Virus promoter (CMP), the CMP short version (CMPS), the Rubisco small subunit promoter, or the maize ubiquitin promoter.

In an embodiment, the plant may be genetically engineered by stable transformation, wherein the nucleic acid encoding the recombinant protein integrates into a genome of the transformed plant. The genetically engineered plant may be created by Agrobacterium-mediated transformation using a vector suitable for stable transformation described herein. The genetically engineered plant may be created by any other methods for transforming plants, for example, particle bombardment, or protoplast transformation via direct DNA uptake. The genetically engineered plant may include any isolated nucleic acids, amino acid sequences, expression cassettes, or vectors herein.

In an embodiment, the plant may be genetically engineered to transiently express the recombinant protein. The term “transient expression” refers to the expression of an exogenous nucleic acid molecule delivered into a cell, e.g., a plant cell, and not integrated in the plant's cell chromosome. Expression from extra-chromosomal exogenous nucleic acid molecules can be detected after a period of time following a DNA-delivery. Virus-based vectors may be used to carry and express exogenous nucleic acid molecules. Virus-based vectors may replicate and spread systemically within the plant. Use of virus based vectors may lead to very high levels of protein accumulation in genetically engineered plants.

In an embodiment, the plant may be genetically engineered to be a conventional mutant having one or more mutations in a nucleic acid sequence encoding a protein involved in regulating levels of a compound or compounds conferring medicinal properties to the plant. The mutations may be deletions, insertions, modifications, or substitutions of nucleic acids in a sequence of the target genes.

The mutant plant may be created by mutagenizing plant seeds, e.g., by chemical mutagenesis (EMS) or radiation, and selecting the mutants by PCR amplification and sequencing the mutant PCR product. The mutant plant may be created by using mutagenesis and screening strategies such as Targeted Induced Lesions In Genomics (TILING), T-DNA insertion and transposon-based mutagenesis.

The mutant plant may be genetically engineered through site directed mutagenesis. See Voytas 2013 Annual Review of Plant Biology 64: 327, which is incorporated herein as if fully set forth. The mutant plant may be genetically engineered through somaclonal variation resulted from exposing plants or plant explants to in vitro tissue culture conditions, e.g., plant growth regulators, auxins or cytokinins. The mutant plant may be, for example, Goldenseal plant with enriched levels of compounds conferring medicinal properties. The mutant Goldenseal plant may include elevated levels of alkaloids, e.g., berberine, β-hydrastine, canadine and canadaline, compared to the levels of such alkaloids observed in non-mutant wild type plants. The mutant plant may be a mutant Echinacea plant. The mutant Echinacea plant may include elevated levels of active ingredients including alkamides, flavonoids, essential oils, and polyacetylenes. The mutant plant may be a mutant Kalanchoe plant. The mutant Kalanchoe plant may have elevated levels of kaempferol and quercetin.

The genetically engineered plant may be a whole plant, or a part of a plant. The part of a plant may be, but is not limited to, a stem, a leaf, a flower, a seed, or a callus. The genetically engineered plant may be a progeny, or descendant of a genetically engineered plant. The genetically engineered plant may be obtained through crossing of a genetically engineered plant and a non-genetically engineered plant as long as it retains the exogenous or modified nucleic acid as described above.

In an embodiment, a method for producing a recombinant protein in a plant. The method may include a step of genetically engineering a plant to include a nucleic acid encoding a recombinant protein. The method may further include culturing the plant under conditions effective for expression of the recombinant protein. The method of genetically engineering the plant may include stably transforming the plant using Agrobacterium-mediated transformation, or transiently expressing the recombinant proteins by methods described herein.

In an embodiment, the method may further include isolating and purifying the recombinant protein.

In an embodiment, the recombinant protein may be any therapeutically effective protein. The term “therapeutically effective protein” refers to a protein capable of generating an appearance of antigen-specific antibodies, such as in serum, or remediation of disease symptoms when applied to a subject in need thereof. The therapeutically effective proteins may be but are not limited to microbicides, vaccines, antibodies, antigens, growth factors, transcription factors, or enzymes.

Therapeutic efficacy and toxicity of active agents in a composition may be determined by standard pharmaceutical procedures, for example, by determining the therapeutically effective dose in 50% of the population (ED50) and the lethal dose to 50% of the population (LD50) in cells cultured in vitro or experimental animals. Plant-derived compositions may be evaluated based on the dose ratio of toxic to therapeutic effects (LD59/ED50), called the therapeutic index, the large value of which may be used for assessment. The data obtained from cell and animal studies may be used in formulating a dosage for human use.

The therapeutic dose shown in examples herein may be at least one microgram (1 μg), or about 3×1 μg, or about 10×1 μg unit of antigen/dose/animal. As plant-based vaccines may be readily produced and inexpensively engineered and designed and stored, greater doses for large animals may be economically feasible. For an animal several orders of magnitudes larger that the experimental animals used in examples herein, the dose may be easily adjusted, for example, to about 3×10×1 μg, or about 3×20×1 μg, or about 3×30×1 μg for animals such as humans and small agricultural animals. However, doses of about 3×40×1 μg, 3×50×1 μg or even about 3×60×1 μg, for example, for a high value zoo animal or agricultural animal such as an elephant, may be provided. For preventive immunization, or periodic treatment, or treatment of a small wild animal, smaller doses such as less than about 3×1 μg, 1 μg and less than about 0.5 μg per dose, may be provided (Portocarrero, 2008 Vaccine 26: 5535, which is incorporated herein by reference as if fully set forth).

In an embodiment, a method of treating a subject against a disease is provided. The method may include genetically engineering a plant to include a nucleic acid encoding a recombinant protein capable of preventing, curing the disease, or eliminating at least one symptom of the disease in the subject. The method may include harvesting the genetically engineered plant.

In an embodiment, the plant genetically engineered to express the recombinant protein may be applied directly, i.e., without or with a little processing, to skin or mucosal surfaces of a subject. The genetically engineered plants may be used as herbal products, i.e., as cut and powdered roots, tinctures, fluid extracts, powdered extract, pharmaceutically processed capsules, tablets, creams, and salves. Medicinal plants may enhance the potency of pharmaceutical compositions, for example, by eliminating irritation, inflammation, sensitization and dryness if such compositions are applied topically or to mucosal surfaces of the recipients.

In an embodiment, the genetically engineered plant, or part of a plant may be included in a first composition. The first composition may be a liquid that include a diluted extract prepared from the genetically engineered plant. The first composition may be herbal tea resultant from extracting genetically engineered plant into water. The herbal tea may be an infusion. The infusion may be the hot water extract of the genetically engineered plant. The herbal tea may be a decoction. The decoction may be the long-term boiled extract of the genetically engineered plant. The first composition may be a tincture. The tincture may be an alcoholic extract of the genetically engineered plant. The extracts of the genetically engineered plants may be liquid extracts. The extracts of the genetically engineered plants may be dry extracts. Dry extracts may be prepared from the tincture that includes the genetically engineered plant which is evaporated into a dry mass. Dry extracts may be further refined to a capsule or tablet. The method may include contacting the subject with a first composition comprising the recombinant plant, or part thereof.

In an embodiment, the method may include isolating the recombinant protein. The method may further include preparing the second composition that includes the isolated recombinant protein. The second composition may be administered in a formulation with an appropriate pharmaceutically acceptable carrier in a desired dosage. The second composition may be administered in liquid dosage forms. Liquid dosage forms may be prepared for nasal administration. Liquid dosage forms for nasal administration may include, but are not limited to, pharmaceutically acceptable emulsions, microemulsions, solutions, and suspensions. Liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils cottonseed, groundnut, corn, germ, olive, castor, sesame oils, glycerol, tetrahydrofurfuryl alcohol, polyethylene glycols and fatty acid esters of sorbitan, and mixtures thereof. Besides inert diluents, the nasal compositions may also include adjuvants. Liquid dosage forms for nasal administration may be aqueous drops, a mist, an emulsion, or a cream. Dosage forms for topical or transdermal administration of the second composition may include ointments, pastes, creams, lotions, gels, powders, solutions, sprays, inhalants, or patches. Ointments, pastes, creams, lotions, gels may contain other natural constituents of plant cells. The other natural constituents of plant cells may have a synergistic effect in treating the subject. Ointments, pastes, creams, lotions, gels may contain vitamins, ethers, oils, or polysaccharides. Powders and sprays may contain recombinant proteins admixed with excipients such as talc, silicic acid, zinc oxide, sulfur, aluminum hydroxide, calcium silicates, polyamide powder, or mixtures of these substances. Sprays may additionally contain customary propellants, for example, chlorofluorohydrocarbons. The recombinant proteins may be admixed under sterile conditions with a pharmaceutically acceptable carrier and any needed preservatives or buffers as may be appropriate.

The second composition for may be formulated for rectal or vaginal administrations and include suppositories. Suppositories may be prepared by mixing of the recombinant proteins with suitable non-irritating excipients or carriers. The excipients may include natural component of plant oils. The excipients may include cocoa butter. The excipients may include natural complex polysaccharides derived from plants. The excipients may derive from Aloe, Yerba santa, or algae. The excipients may be a conventional polyethylene glycol or a suppository wax. The carriers may be solid at ambient temperature but liquid at body temperature and therefore melt in the rectum or vaginal cavity and release the recombinant proteins.

The method may include contacting the subject with the second composition comprising the recombinant protein.

As used herein, the term “subject” refers to a mammal. A subject may be male or female mammal. A mammal may be an animal or a human. The term “subject” does not denote a particular age. Thus, both adult and newborn individuals are intended to be covered. The compositions described herein are intended for use in any of the above subjects, since the immune systems of all of these subjects operate similarly.

In an embodiment, the composition may be therapeutically effective. Therapeutic efficacy may depend on effective amounts of active agents and time of administering necessary to achieve the desired result. Active agents may be recombinant proteins. Administering a composition may be a prophylactic or preventive measure. Administering of a composition may be a therapeutic measure to promote immunity to the infectious agent, to minimize complications associated with the slow development of immunity especially in patients with a weak immune system, elderly or infants. The plant-derived composition may be provided in a “therapeutically effective amount,” i.e., the amount sufficient to generate appearance of antigen-specific antibodies in serum, or disappearance of disease symptoms. Disappearance of disease symptoms may be assessed by a decrease of virus in feces or in bodily fluids or in other secreted products.

The exact dosage of the composition may be chosen based on a variety of factors and in view of individual characteristics of subjects. Dosage and administration may be adjusted to provide sufficient levels of the active agent or agents or to maintain the desired effect. For example, factors which may be taken into account include the type and severity of a disease; age and gender of the subject; drug combinations; and an individual response to therapy. The active agent may be a recombinant protein. The recombinant protein may be a microbicide, an antigen or an antibody.

In an embodiment, the compositions described herein may be administered using any amount and any route of administration effective for generating an antibody response. The compositions that include medicinal plants may be applied topically to skin or to mucosal surfaces of a subject. A mucosal route may include administering plant-derived composition to any mucosal surface of the body of the subject. Mucosal surfaces may include oral, lingual, sublingual, intranasal, ocular, vaginal, urethral and rectal surfaces. Mucosal administration differs from “systemic” or “parenteral” administration. Systemic administration may include administering compositions to a non-mucosal surface, e.g., intraperitoneal, intramuscular, cutaneous, sub-, or transcutaneous, intra- or transdermal, or intravenous administration.

In an embodiment, Goldenseal-derived compositions comprising Goldenseal plants or components of the Goldenseal plants may be used for treating a variety of diseases including, but not limited to, colds, whooping cough, pneumonia, chronic constipation, hepatic congestion, cerebral engorgements, leucorrhea, and gallstones. Goldenseal-derived compositions may be also applied for treatment of digestive disorders, peptic ulcers, gum diseases, sinusitis, catarrhal deafness, tinnitus, and pelvic inflammatory disorders, along with a variety of other diseases. The Goldenseal properties may allow use of goldenseal-derived compositions to treat vaginal infection, eczema, conjunctivitis, eliminate irritation, inflammation, sensitization and dryness.

In an embodiment, Echinacea-derived compositions comprising Echinacea plants or components of the plants may be used for treating a variety of diseases including, but not limited to, infections, urinary tract infections, vaginal infections, ear infections, wounds, skin infections, inflammatory skin conditions, fever, malaria, and blood poisoning.

In an embodiment, Kalanchoe-derived compositions comprising Kalanchoe plants or parts of Kalanchoe plants may be used for treating various respiratory conditions, such as asthma, coughs and bronchitis. Kalanchoe may be used for treating rheumatism, inflammation, gastric ulcers, infections, and pain. A Kalanchoe leaf infusion or juice may be used for treatments of headaches, toothaches, earaches, eye infections, wounds, ulcers, boils, burns and insect bites. Kalanchoe preparations may be used in surgical, stomatological, and obstetric-gynecological practice

In an embodiment, Calendula-derived compositions comprising Calendula plant or parts of Calendula plants may be used for treating upset stomach, ulcers, hemorrhoids, inflammations, and wounds.

In an embodiment, Thyme-derived compositions comprising Thyme plants or parts of Thyme plants may be used treating coughs, bronchitis, and inflammation of upper respiratory membranes.

The compositions herein may be used to treat or prevent a disease or an abnormal condition in a subject. An “abnormal condition” refers to a function in the cells and tissues in a body of a subject that deviates from the normal function in the body. An abnormal condition may refer to a disease. The disease may be but is not limited to acquired immunodeficiency syndrome (AIDS), anthrax, smallpox, SARS avian flu, hepatitis B, DTP, RSV, papillomavirus, and cancer.

In an embodiment, a plant-derived composition expressing microbicides may be administered to a subject to protect the subject against development of AIDS.

In an embodiment, a plant-derived immunogenic composition expressing a Vaccinia glycoprotein B5 membrane antigen may be administered for immunizing subjects for resistance against poxvirus associated illnesses. See Golovkin et al. 2007 Proc Natl Acad Sci USA 104: 6864, which is incorporated herein as if fully set forth.

In embodiments, treatments of a variety of infectious diseases arising from infection with Vaccinia virus, variola virus, monkeypox virus, raccoon poxvirus, skunk poxvirus, camelpox virus, ectromelia virus, cowpox virus, taterapox virus and volepox virus are provided.

In an embodiment, administering of a plant-derived therapeutic composition may be a preventive treatment of subjects to promote emergency post-infection prophylaxis of a contact with the infectious agent. Administering of a plant-derived composition may be a therapeutic measure for neutralization anthrax toxin produced by bacterial pathogen Bacillus anthracis and minimizing complications associated with accumulation of deadly toxin in patients infected with the pathogen bacteria. Administering the plant-derived composition may be used for treatment of a variety of, symptoms and consequences of various forms of anthrax disease arising from infection with pathogenic bacteria Bacillus anthracis. Plant-derived therapeutic compositions may be useful to treat patients being in contact with anthrax toxin, pathogen, infected animal or human, belonging to a group of risk of biological weapon attack.

In an embodiment, the method may further include measuring cell viability in the presence of different concentrations of the anthrax toxin, wherein cell viability is a percentage of surviving cells protected by the antitoxin in comparison to the complete lysis in the control.

In an embodiment, the method may further include measuring survival of animals after challenging with lethal concentration of the anthrax toxin or B. anthracis spores, followed by administration of protective amounts of the recombinant antitoxin. The survival may be a percentage of live animals protected by the antitoxin in comparison to unprotected objects in the control.

In an embodiment, a method of propagating a plant in vitro is provided. The plant may belong to the genus selected from the group consisting of Hydrastis, Echinacea, Thymus, Calendula, and Kalanchoe. The method may include culturing a plant, or part of the plant on a culture medium. The culture medium may include one or more plant growth regulators. The method includes recovering multiple shoots grown from the plant or the part of the plant. As used herein, the term “plant growth regulators” or “plant hormones” refers to chemicals or a group of chemicals that are used in plant cell culture media to facilitate plant growth.

In an embodiment, the plant growth regulator may be a cytokinin. The cytokinin may be but is not limited to kinetin, benzylaminopurine (BAP), zeatin, and thidiazuron. Cytokinins are known to induce shoot formation from plant explants.

In an embodiment, the plant growth regulator may be an auxin. The auxin may be, but is not limited to, indole-butyric acid (IBA), indole-acetic acid (IAA), naphthalene acetic acid (NAA) and 2,4-dichlorophenoxy-acetic acid (2,4-D). Auxins may be combined with cytokinins to induce shoot formation from plant explants. Auxins may facilitate callus formation from plant explants. A culture medium may have combinations of auxins and cytokinins in different concentrations during different stages of shoot development. Multiple shoots may be produced on the culture medium.

In an embodiment, shoots may be excised. The excised shoots may be further rooted in a rooting medium. The rooting medium may be a hormone free-medium. The excised shoots may be rooted in a medium that includes an auxin. The excised shoots may be rooted in any other way. For example, shoots may be rooted in soil under the greenhouse conditions.

In an embodiment, a method of producing a cell suspension culture derived from a plant, part or tissue thereof is provided. The method may include culturing plant, part or tissue thereof in a liquid medium. The liquid medium may include an auxin. The auxin may be but is not limited to IBA, IAA, NAA and 2,4-D. The auxin may be 2,4-D. The cell suspensions cultures may be kept in the dark. The cell suspension cultures may be agitated during culturing. The cell suspension cultures may be further used for propagation of the plants.

In an embodiment, in vitro propagation methods and tissue culture may be developed for mass multiplication of many important medicinal plants. Development of efficient tissue culture protocols for Goldenseal, Echinacea, Thymus, Calendula, and Kalanchoe, may allow production of sufficient plant material for commercial purposes. Efficient mass propagation, both, as whole plants, and as cell cultures may be established and may serve as a basis for the development of a protocol for genetic transformation. Plants produced by in vitro propagation may be characterized by a uniform quality and significant increase in biomass yields.

Further embodiments herein may be formed by supplementing an embodiment with one or more element from any one or more other embodiment herein, and/or substituting one or more element from one embodiment with one or more element from one or more other embodiment herein.

The following non-limiting examples are provided to illustrate particular embodiments. The embodiments throughout may be supplemented with one or more detail from one or more example below, and/or one or more element from an embodiment may be substituted with one or more detail from one or more example below.

### Example 1

### Strategy for Production of Recombinant Proteins in Medicinal Plants

Due to concerns raised about producing industrial and pharmaceutical proteins in food and feed crops, efforts to develop tissue culture and transformation procedures for recalcitrant plants, especially medicinal plants, are renewed. Properties of medicinal plants to be used as factories for producing recombinant proteins may add value and enhance benefits of pharmaceutical compositions produced from these plants if the plants or parts thereof are included in the compositions. Thus, the benefits of producing pharmaceutical proteins in medicinal plants may outweigh the hurdles associated with the development of tissue culture and transformation methods for these plants.

For example, medicinal plant Goldenseal (Hydrastis canadensis), a member of the Ranunculaceae family, has been traditionally used for treating a variety of diseases and illnesses such as whooping cough, pneumonia, chronic constipation, hepatic congestion, cerebral engorgements, leucorrhea, and gallstones. Goldenseal is considered effective for treating the mucosal surfaces lining the mouth, throat, intestines, stomach, urinary tract, vagina and rectum. See Foster and Tyler 1999 Tyler's Honest Herbal: A sensible guide to the use of herbs and related remedies. Binghamton, N.Y., The Haworth Herbal Press.

Goldenseal is a small perennial herb, native to southeastern Canada and the northeastern US. Goldenseal's popularity has led to overharvesting in the wild. The Convention on International Trade in Endangered Species of Wild Fauna and Flora (CITES), Appendix II, lists the plant as an endangered species. Development of tissue culture techniques for mass propagation is desired to restore the population of the plant and for commercial uses. In addition, commercial preparations of wild harvested goldenseal might contain environmental pollutants, particularly, the heavy metals. See Liu et al. 2004 In Vitro Cell Dev Biol Plant 40:75. Plants grown in vitro evade the problem of environmental pollutants, microbial infestations, and soil-born contaminants. See Saxena 2001 Plant Cell Tiss Org Cult 62:167. Literature provides very limited information regarding tissue culture propagation of goldenseal and no reports on genetic transformation of the plant. See Liu et al. 2004 In Vitro Cell Dev Biol Plant 40:75; Hall and Camper 2002 In vitro Cell. Dev. Biol. Plant 38: 293; Bedir et al. 2003 Planta Med 69:86; and He et al. 2007 Sci Hort 113: 82.

Echinacea (Echinacea purpurea), a member of Asteraceae family, is a perennial herb native to the Midwestern region of the U.S.A. Historically, Echinacea had been used by Native Americans to treat infections and wounds, fever, malaria, blood poisoning, syphilis and diphtheria. Echinacea is known to alleviate symptoms of the common cold and flu, such as sore throat, cough, and fever, and shorten the duration of the disease. The active ingredients of Echinacea purpurea include caffeic acid derivatives, alkamides, flavonoids, essential oils, and polyacetylenes. Because of its medicinal properties, Echinacea is an attractive plant for mucosal and topical applications, and can be used alone or in formulations for treating vaginal and urinary tract infections, ear infections, for healing wounds, burns, skin infections and inflammatory skin conditions.

Another medicinal plant, Kalanchoe (Kalanchoe pinnata), is a member of the Crassulaceae family, that is cultivated in the U.S. as an ornamental plant. Kalanchoe had a history of use for treating various respiratory conditions, such as asthma, coughs and bronchitis. Kalanchoe use was reported for treating rheumatism, inflammation, gastric ulcers, infections, and pain. A leaf infusion or juice is used for treatments of headaches, toothaches, earaches, eye infections, wounds, ulcers, boils, burns and insect bites. Kalanchoe preparations are used in surgical, stomatological and obstetric-gynecologic practice, and reported to be efficient for topical and mucosal applications including oral, intranasal, vaginal, and rectal application. Coumaric, ferulic, syringic, caffeic and phydroxybenzoic acids, kaempferol and quercetin were detected in Kalanchoe leaves.

Calendula (Calendula officinalis), an annual plant, native to Mediterranean countries, belongs to the Asteraceae family. Traditionally, Calendula has been used to treat stomach upset, ulcers, hemorrhoids, inflammations, and wounds. The flower petals of the calendula plant have been used for medicinal purposes since at least the 12th century. The anti-inflammatory effects have been reported to be due to the triterpenoids, and specifically faradiol, found in calendula. Calendula preparations are used for healing wounds, including traumatic wounds and chronic wounds, such as pressure sores and diabetic ulcers. Calendula includes high levels of flavonoids, plant-based antioxidants that protect cells from free radicals. Calendula preparations are applied to the skin to help burns, bruises, and cuts heal faster, and to fight the minor infections they cause. Calendula preparations are efficiently using for mucosal applications, particularly, for the oral and pharyngeal mucosa, as well as for vaginal and rectal mucosal surfaces.

Thyme (Thymus vulgaris), a perennial shrub native to the Mediterranean, belongs to the Lamiaceae family. Thyme has been reported for medicinal uses for thousands of years. Traditional uses of thyme include treatments of coughs, bronchitis, and catarrh, i.e., inflammation of upper respiratory tract mucous membranes. Thyme essential oil contains a range of compounds, such as p-Cymene, myrcene, borneol and linalool. Thyme oil is also reported to contain 20-54% of thymol. The Thyme flowers, leaves, and oil are used in herbal medicine. Topically, Thyme has been used for bald patches, laryngitis, tonsillitis, and mouth inflammation, and Thyme preparations are used for topical and mucosal applications.

There is a demand for products derived from medicinal plants but only a limited supply of these plants in the wild. The data herein demonstrates the technologies for mass propagation of the medicinal plants that permit the production of Hydrastis, Echinacea, Thymus, Calendula, and Kalanchoe plants that may be free from environmental contamination and human pathogens.

Additionally, the tissue culture technologies developed herein may be useful for development of improved lines of medicinal plants enriched in valuable compounds and characterization of these compounds, such as alkaloids hydrastine and berberine of Goldenseal, alkamides, flavonoids, essential oils, and polyacetylenes of Echinacea and kaempferol and quercetin of Kalanchoe. With respect to development of somaclonal variants of medicinal plants with increased content of valuable compounds, several tissue culture approaches were utilized including in vitro propagation, callus initiation, cell suspension, and plant regeneration.

Transformation systems for medicinal plants have been developed including stable transformation and transient expression. Transgenic Hydrastis, Echinacea, Thymus, Calendula, and Kalanchoe plants with high expression of recombinant proteins have been produced. For stable transformation of Hydrastis, Echinacea, Thymus, Calendula, and Kalanchoe Agrobacterium-mediated methods were used as well as particle bombardment technology. These technologies may be used for other medicinal plants.

Medicinal plants can be used for production and direct delivery of commercial, industrial, cosmetic and pharmaceutical proteins including microbicides, vaccines, antibodies and many others. Moreover, due to valuable medicinal properties, Goldenseal, Echinacea, Calendula, Kalanchoe and Thyme are attractive plant systems for mucosal application. Preparations of these medicinal plants can be applied directly without purification. Data herein open new opportunities to utilize medicinal plants as a platform for production and delivery of recombinant proteins.

### Example 2

### Plant Material and In Vitro Cultures

Seeds and leaf tissues of medicinal plants Goldenseal, Echinacea, Kalanchoe, Thyme and Calendula have been sterilized and cultured in vitro. For development of an efficient regeneration system, cotyledons and leaf segments of these medicinal plants were placed into 100×15 mm Petri dishes containing 25 ml of MS medium supplemented with plant hormones (Murashige and Skoog 1962 Physiol Plant 15: 473). Various concentrations of cytokinins: 6-bezylaminopurine (BAP: 0.5, 1, and 2 mg/l), kinetin (0.5-1 mg/l), zeatin (0.5-1 mg/l), thidiazuron (TDZ; 0.5, 1 and 2 mg/l), and auxins: naphtaleneacetic acid (NAA; 0.1, 0.2, 0.3, and 0.5 mg/l) and 2,4-dichlorophenoxyacetic acid (2,4-D; 0.1-0.2 mg/l) were tested for shoot induction. Typically, ten explants were plated per Petri dish, cultured for 7-8 weeks, and analyzed for shoot regeneration efficiency assessed as the percentage of explants producing shoots per total number of explants plated.

Plant material from all medicinal plants was cultivated at 24° C. at 16 h-light/8 h-dark photoperiods and light intensity of 40 E/m2/S1.

Goldenseal (Hydrastis Canadensis). Goldenseal rhizomes were obtained from North Carolina Goldenseal and Ginseng Company (Marshall, N.C.) and transplanted into the Pro-Mix BX potting soil (Premier Tech Horticulture Company, Quakertown, Pa.). Leaf explants were excised from the 1-2 month-old plants, and surface sterilized by immersion in 70% ethanol for 1 min, followed by soaking in 1.5% sodium hypochlorite for 4-6 min. After rinsing 3 times in sterile distilled water and blotting dry with the sterile filter paper, 0.7 cm² leaf segments were transferred onto the following MS-based regeneration media described in Table 1: MSR1 (1 mg/l BAP and 0.1 mg/l NAA), MSR2 (1 mg/l BAP; 1 mg/l TDZ and 0.2 mg/l NAA) and MSR3 (1 mg/l BAP; 0.5 mg/l kinetin and 0.3 mg/l NAA).

TABLE 1
Media for tissue culture

| Name | Media composition |
|---|---|
| MS | Basic MS basal medium with 3% sucrose, 0.7% agar |
| MSR1 | MS with 1 mg/l BAP, 0.1 mg/l NAA, 3% sucrose, 0.7% agar |
| MSIR | MS with 3% sucrose, 0.7% agar, 1 mg/l IBA |
| MSR2 | MS with 1 mg/l BAP, 1 mg/l TDZ, 0.2 mg/l NAA, 3% sucrose, |
| | 0.7% agar |
| MSR3 | MS with 1 mg/l BAP, 0.5 mg/l kinetin, 0.3 mg/l NAA 3% |
| | sucrose, 0.7% agar |
| MSR4 | MS with 0.4 mg/l BAP, 3% sucrose, 0.7% agar |
| MSR5 | MS with 1 mg/l BAP, 0.5 mg/l zeatin 0.2 mg/l NAA, 3% |
| | sucrose, 0.7% agar |
| MSR6 | MS with 2 mg/l BAP, 0.1 mg/l NAA, 3% sucrose, 0.7% agar |
| MSR7 | MS with 1 mg/l BAP, 1 mg/l TDZ, 0.3 mg/l NAA, 3% sucrose, |
| | 0.7% agar |
| MSC1 | MS with 2 mg/l NAA, 1 mg/l 2,4-D, 0.5 mg/l BAP, 3% sucrose, |
| | 0.7% agar |
| MSC2 | MS with 1 mg/l 2,4-D, 3% sucrose, 0.7% agar |
| MSC3 | MS with 0.5 mg/l NAA, 1 mg/l 2.4-D, 0.5 mg/l BAP, 3% |
| | sucrose 0.7% agar |
| MST2 | MSR3 with 300 mg/l timentin |
| MST3 | MSR3 with 300 mg/l timentin, 20 mg/l kanamycin |
| MST4 | MSR4 with 300 mg/l timentin, 30 mg/l kanamycin |
| MST5 | MS with 1 mg/l IBA, 30 mg/l kanamycin, 100 mg/l timentin |
| MST6 | MSR6 with 300 mg/l timentin, 50 mg/l kanamycin |
| MST7 | MSR7 with 300 mg/l timentin, 30 mg/l kanamycin |
| MST8 | MSR7 with 300 mg/l timentin, 50 mg/l kanamycin |
| MSS | MS with 1 mg/l 2.4-D, 3% sucrose |

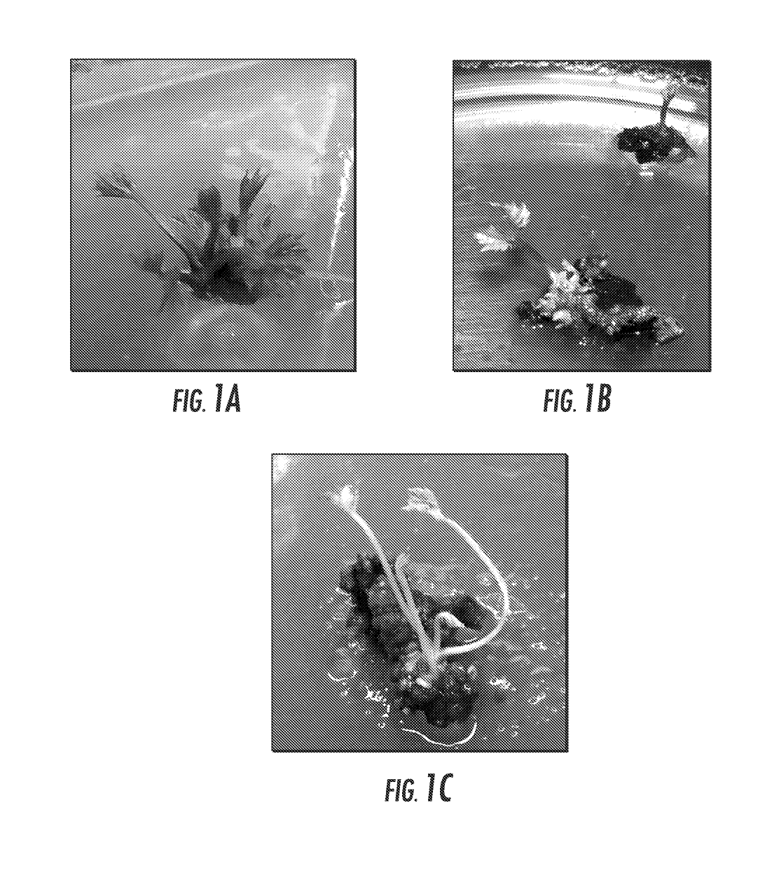

Shoot development was observed after 5-8 weeks of culture (FIGS. 1A-1B). The highest regeneration efficiency of 68% was observed for explants cultured on MSR3 medium. For further shoot development, explants were transferred to the elongation medium. FIG. 1C illustrates Goldenseal shoots growing on the MSR4 elongation medium (0.4 mg/l BAP). For rooting, well developed shoots were transferred to MS media was supplemented with NAA, indole-3-acetic acid (IAA), indole-3-butyric acid (IBA), or no hormones. The best root development was observed on the MS medium supplemented with 1 mg/l IBA. Rooted plants were transferred to the Pro-Mix soil (Premier Tech Horticulture Company, Quakertown, Pa.). Morphology of the plants propagated in vitro was normal, that is, similar to that of the wild type plants.

Echinacea (Echinacea purpurea), Calendula (Calendula officinalis), Kalanchoe (Kalanchoe pinnata) and Thyme (Thymus vulgaris). Seeds of Echinacea, Calendula and Thyme were obtained from Horizon Herbs Co. (Williams, Oreg.). All seeds were sterilized with 70% ethanol for 1 min followed by soaking in 1.5% sodium hypochlorite: for 10 min for Echinacea and Calendula, and 5 min for Thymus. After washing with sterile distilled water, 10-15 seeds of each medicinal plant were placed into the Magenta GA-7 box containing 40 ml of the MS-based germination medium supplemented with 10 g/l sucrose and 7 g/1 agar. Stem segments with axillary buds were sub-cultured onto fresh MS media every 7-8 weeks.

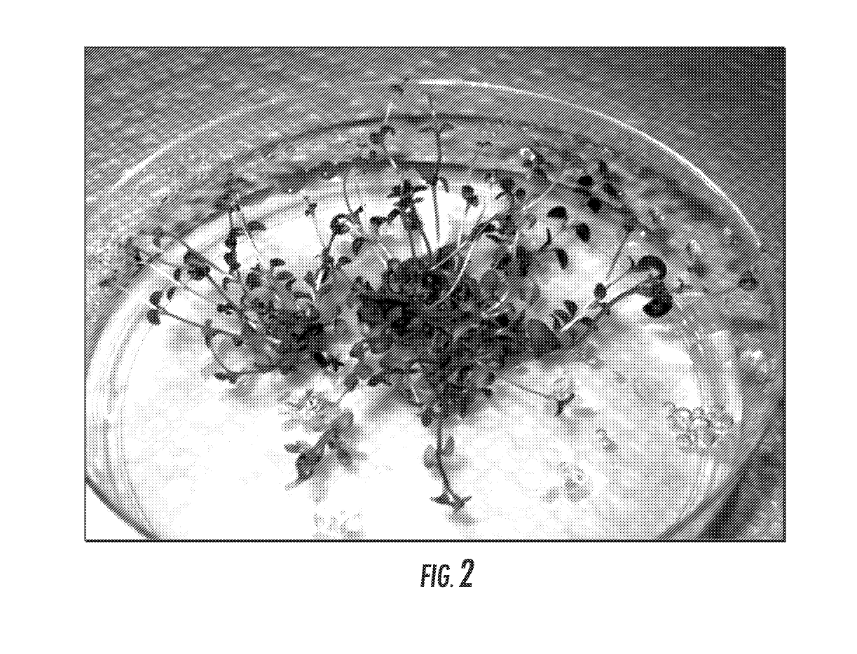

Thymus vulgaris. Leaves of the in vitro propagated 2 month-old plants were cut into 2-3 mm pieces and plated onto regeneration media. FIG. 2 illustrates multiply shoot regeneration on MSR3 medium. The highest shoot regeneration efficiency of 36% was observed on MSR3 medium. See Table 1.

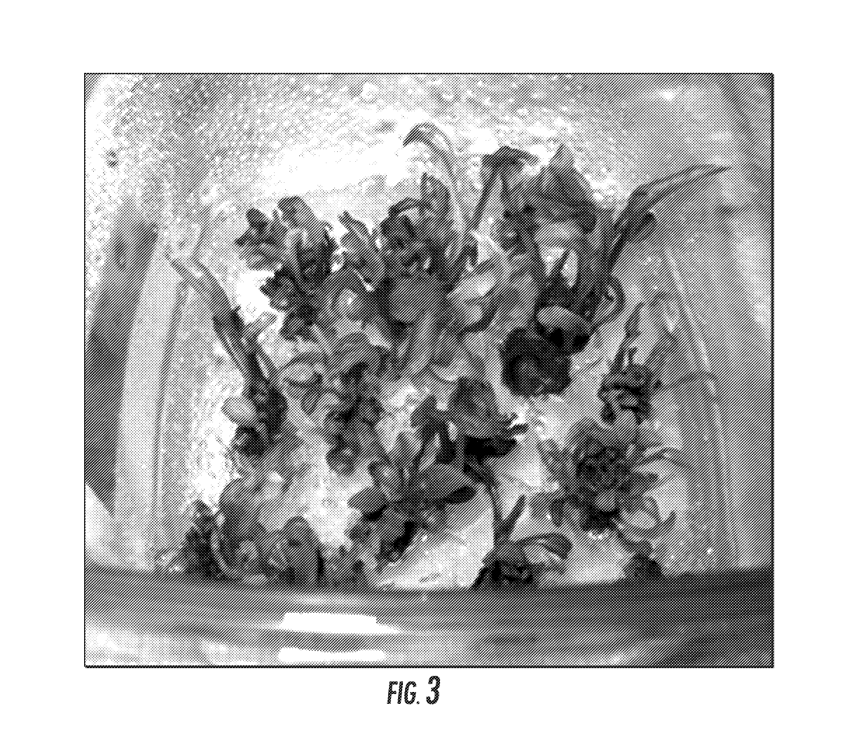

Echinacea purpurea. For regeneration, 10 day-old cotyledons and leaves from 2-month-old plants were cut into 4-5 mm explants and plated onto the regeneration medium. FIG. 3 illustrates multiple shoots were produced from Echinacea explants after 5-6 weeks of culture. The highest regeneration efficiency of 84% and 81% was observed for MSR5 (1 mg/l BAP, 0.5 mg/l zeatin, 0.2 mg/l NAA) and MSR7 (1 mg/l BAP, 1 mg/l thidiazuron, 0.3 mg/l NAA), respectively. See Table 1. It was noticed that the cotyledon explants produced 2.5 times more shoots compare to the leaf explants.

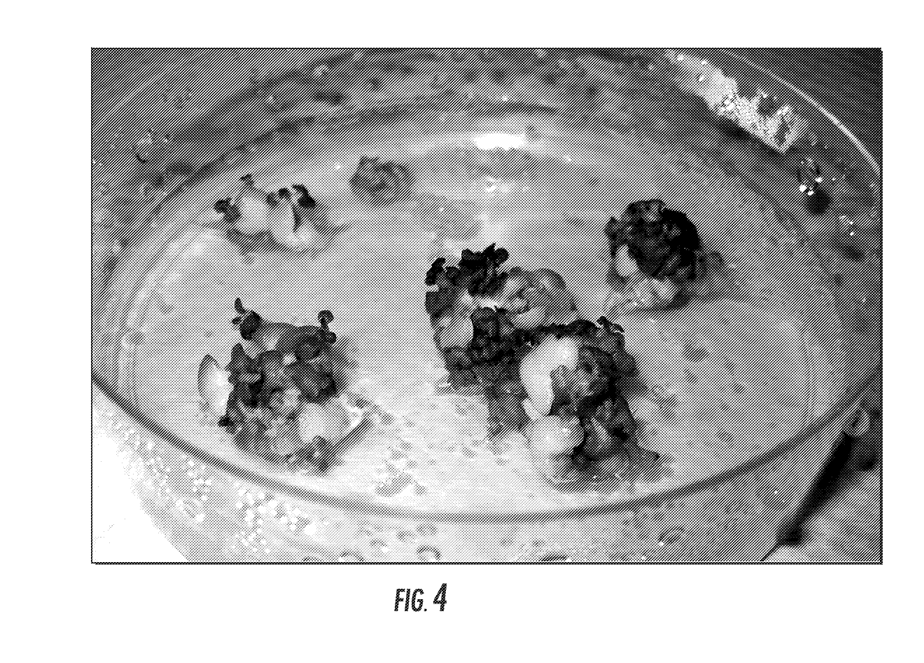

Kalanchoe pinnata. Fresh Kalanchoe leaves were obtained from Tropilab Inc. (St. Petersburg, Fla.). Leaves were surface sterilized by immersion in 70% ethanol for 1 min, followed by soaking in 1.5% sodium hypochlorite for 8 min. After rinsing 3 times in sterile distilled water and blotting dry with the sterile filter paper, 1×1 cm leaf segments were transferred to the MS-based regeneration medium. The highest regeneration efficiency of 79% was observed on the MSR6 medium containing 2 mg/l BAP, 0.1 mg/l NAA, 30 g/l sucrose and 7 g/l agar (Table 1). The regenerated shoots were excised and transferred to MS medium. Shoots formed roots and produced whole plants within 3-5 weeks. For propagation of Kalanchoe, stem segments with axillary buds were transferred to the fresh MS medium supplemented with 30 g/l sucrose and 7 g/l agar every 2 months. FIG. 4 illustrates development of multiple shoots from Kalanchoe leaf explants.

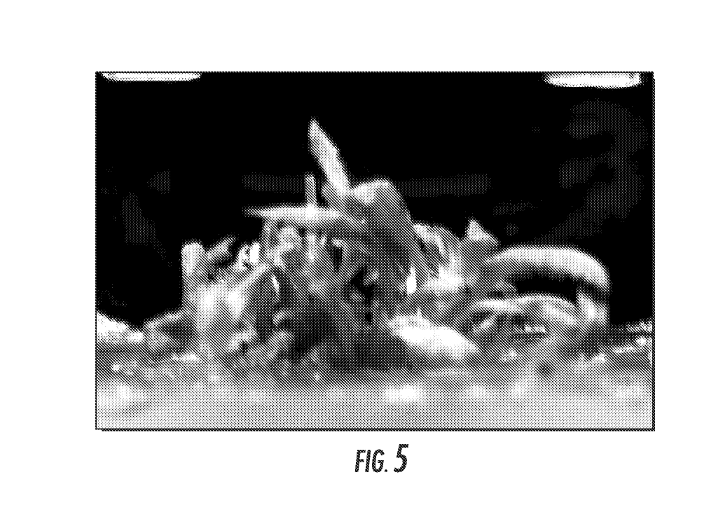

Calendula officinalis. Cotyledons and leaves from the 1 month-old-plants were cut into 4-5 mm pieces and tested for regeneration capacity. FIG. 5 illustrates shoot regeneration from Calendula. The best shoot regeneration of 48% was observed on MSR3 medium. See Table 1.

### Example 3

### Induction of Callus and Cell Suspension

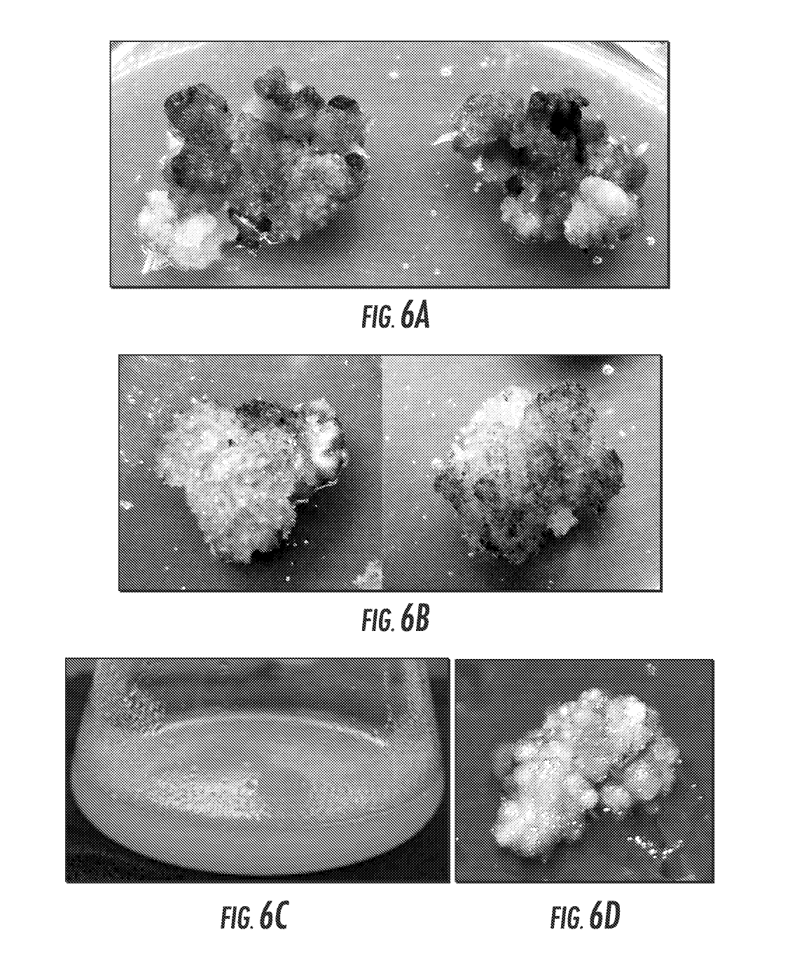

A phenomenon of somaclonal variability is known to be induced by in vitro conditions for callus tissues or cell suspensions (Brown and Thorpe 1995 World J Microbiol Biotechnol 11: 409; Larkin and Scowcroft 1981 Theor Appl Genet 60: 197). For development of somaclonal variants of medicinal plants with enhanced medicinal properties, callus cultures and cell suspensions were initiated. Leaf segments of each of Goldenseal, Echinacea, Kalanchoe, Thyme and Calendula were placed into the 100×15 mm Petri dishes containing MS-based callus induction medium. Plates were incubated in the dark at 24° C. for 6-7 weeks. The following plant growth regulators were tested for callus induction: NAA (1-2 mg/l), BAP (0.3-0.5 mg/l) and 2.4-D (0.5-1 mg/l). The highest percentage of callus tissues for Goldenseal (48%), Echinacea (72%) and Kalanchoe (42%) was produced on the MSC1 medium See Table 1. FIG. 6A illustrates the Goldenseal callus grown on the MSC1 medium. For callus induction from Goldenseal explants, MSC1 medium was observed to be the most efficient. Well-developed Goldenseal calli were selected and transferred to the callus propagation MSC2 medium (Table 1) and incubated in the dark. FIG. 6B illustrates callus grown on MSC2 medium. After several passages on the MSC2 medium, friable callus was produced and used for initiation of cell suspensions. For this, approximately 1 g of fresh callus tissue was transferred into the sterile 250 ml conical flasks containing 50 ml of liquid MSS medium (Table 1). Cell cultures were grown in the dark at 25° C., on a rotary shaker. Maintenance of the cell suspension was carried out on the MSS medium with 10-14 day intervals for subcultures. FIG. 6C illustrates the Goldenseal cell suspensions. FIG. 6D illustrates shoot regeneration from callus tissues after 8 weeks of culturing on MSR3 medium.

### Example 4

### Agrobacterium-Mediated Transformation

For development of an efficient transformation protocol for medicinal plants, the pBI121 vector containing the reporter GUS gene under control of the CaMV 35S promoter and the nptII gene under control of the NOS promoter were used (Jefferson et al. 1987 EMBO J 6: 3901). The pBI121 was introduced into the Agrobacterium tumefaciens strain LBA4404. Agrobacteria were grown at 28° C. on solid LB media supplemented with 50 mg/l kanamycin and 20 mg/l rifampicin. The bacterial cell suspension was prepared by inoculating 20 ml of the liquid LB medium with a single bacterial colony and grown for 1-2 days at 150 rpm in a shaker. The suspension of Agrobacterium was diluted with a liquid MS medium to obtain OD₆₀₀ 0.5, 0.3 and 0.1.

Leaf segments of a medicinal plant were then incubated with Agrobacterium suspension (OD₆₀₀ 0.5, 0.3 or 0.1) for 10 min. For Echinacea and Kalanchoe, OD₆₀₀ 0.5 was found the best for high transformation efficiency. For the Goldenseal, Calendula and Thyme explants, inoculation with Agrobacterium suspension diluted to OD₆₀₀ 0.1 resulted in the highest number of transformed plants. After blotting dry with the sterile filter paper, the inoculated explants were transferred to the MS co-cultivation medium supplemented with 100 μM acetosyringone, and incubated in the dark for 2-3 days at 24° C. It was further observed that 2 days of co-cultivation resulted in the highest transformation efficiency.

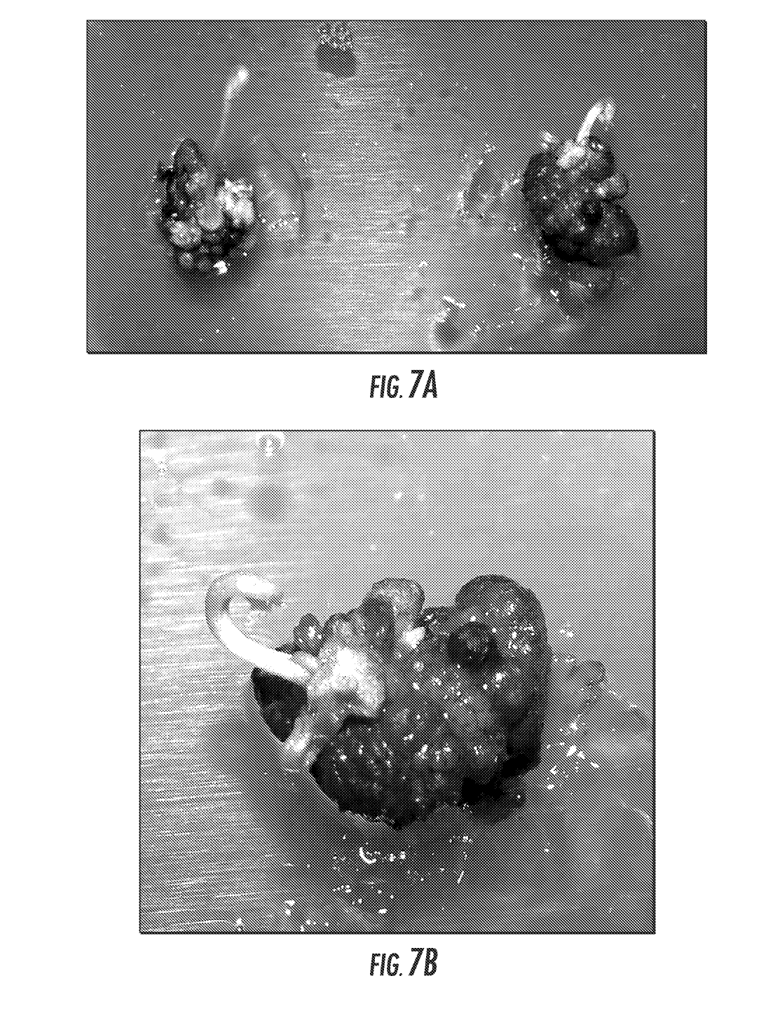

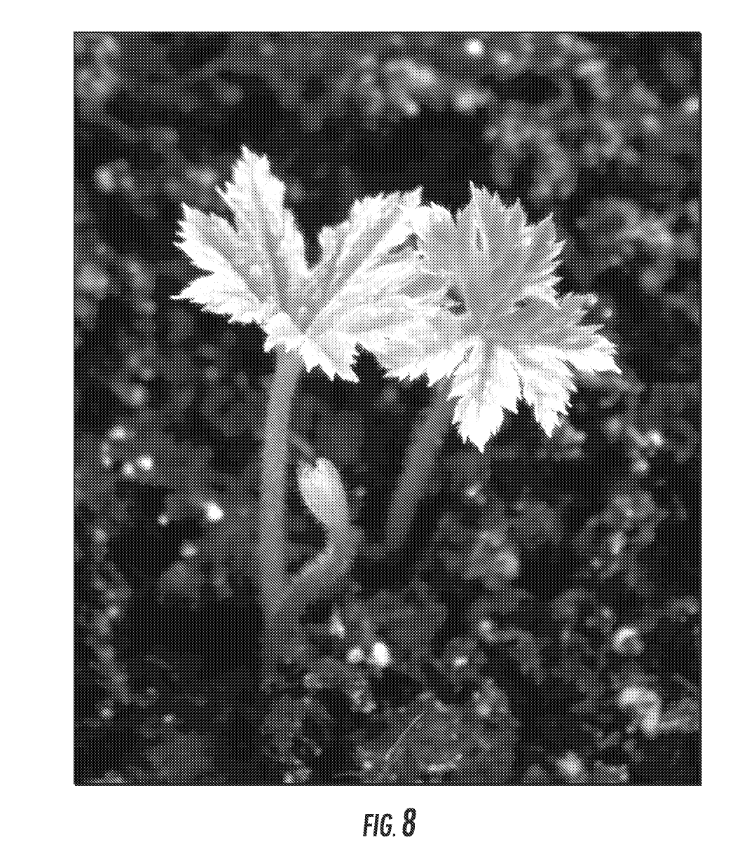

Several selection schemes were tested. Only Kalanchoe transgenic plants were produced when selection was initiated immediately after co-cultivation. For Goldenseal, Echinacea, Thyme and Calendula, no transgenic plants were recovered when selection started immediately after co-cultivation. For these plants, selection was delayed. During the delay period, explants were kept on the MST2 delay medium supplemented with timentin to eliminate Agrobacterium but containing no selection agent. Delay periods of 10, 15 and 20 days were tested. The highest rate of regeneration of transgenic shoots was observed for leaf explants cultured on the MST2 medium for 20 days before transfer to the first selection medium. After the delay period, Goldenseal, Thyme and Calendula explants were transferred to the first selection MST3 medium supplemented with 20 mg/l kanamycin, and after 3-4 weeks transferred to the second selection medium MST4 supplemented with 30 mg/l kanamycin. Figures FIGS. 7A-7B illustrate effective selection of the Goldenseal transformants on the MST4 medium. FIG. 7A illustrates development of the transgenic shoots on the MST4 medium in the presence of 30 mg/l of kanamycin. FIG. 7B illustrates the magnified shoot from FIG. 7A. FIG. 8 illustrates the morphologically normal transgenic Goldenseal plants recovered from transformation and selection.

For selection of the transgenic Echinacea plants, the first regeneration selection medium was MST7 supplemented with 30 mg/1 kanamycin, and the second selection medium was MST8 supplemented with 50 mg/l kanamycin (Table 1). The Echinacea explants were transferred to MST8 medium, after 10-14 days of selection on the MST7 medium.

For selection of Kalanchoe, the regeneration selection medium was MST6 supplemented with 50 mg/l kanamycin (Table 1). No delay of selection was used for Kalanchoe.

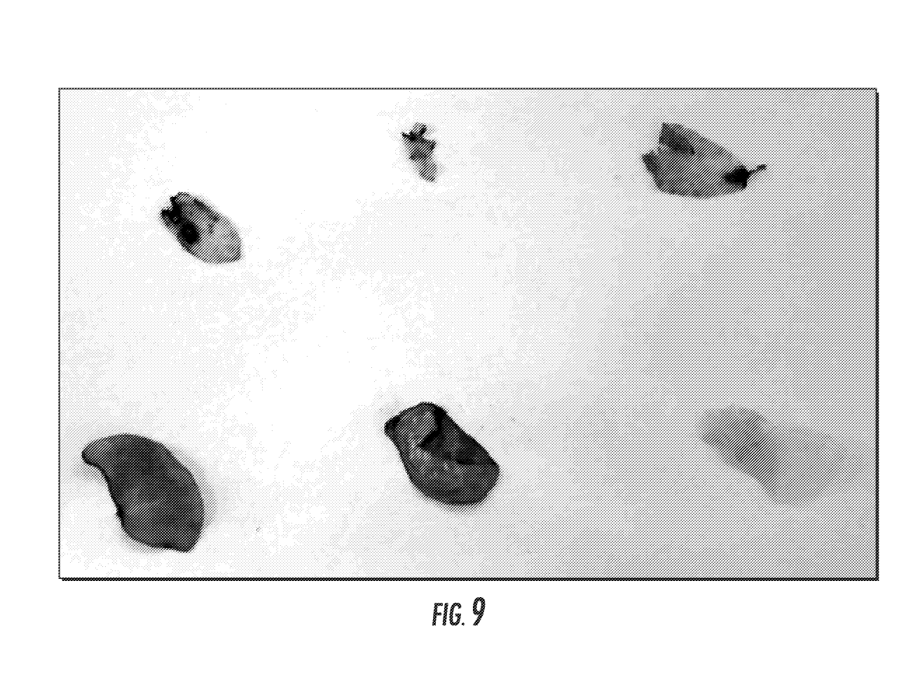

The kanamycin-resistant shoots of medicinal plants, 2-4 cm in length, developed on the selection media were excised and transferred to the root induction MST5 medium supplemented with 30 mg/l kanamycin for Goldenseal, Thyme and Calendula, or the MS medium supplemented with 50 mg/l kanamycin for Echinacea and Kalanchoe. Plantlets with roots were subsequently transferred to pots containing the Pro-Mix soil. Putative transgenic medicinal plants were tested for GUS expression by histochemical staining. FIG. 9 illustrates histochemical GUS analysis of the transgenic Calendula plants. GUS activity in the transgenic Calendula plants was observed as the blue staining. Referring to FIG. 9, the leaf segments from the transgenic plant developed blue staining while the leaf segment from the non-transgenic control plants were bleached (shown on the right). More than 95% of the plants produced after 2 rounds of selection on kanamycin were observed to be GUS-positive, and thus, confirmed transformants (Jefferson et al. 1987 Plant Mol Biol Rep 5: 387).

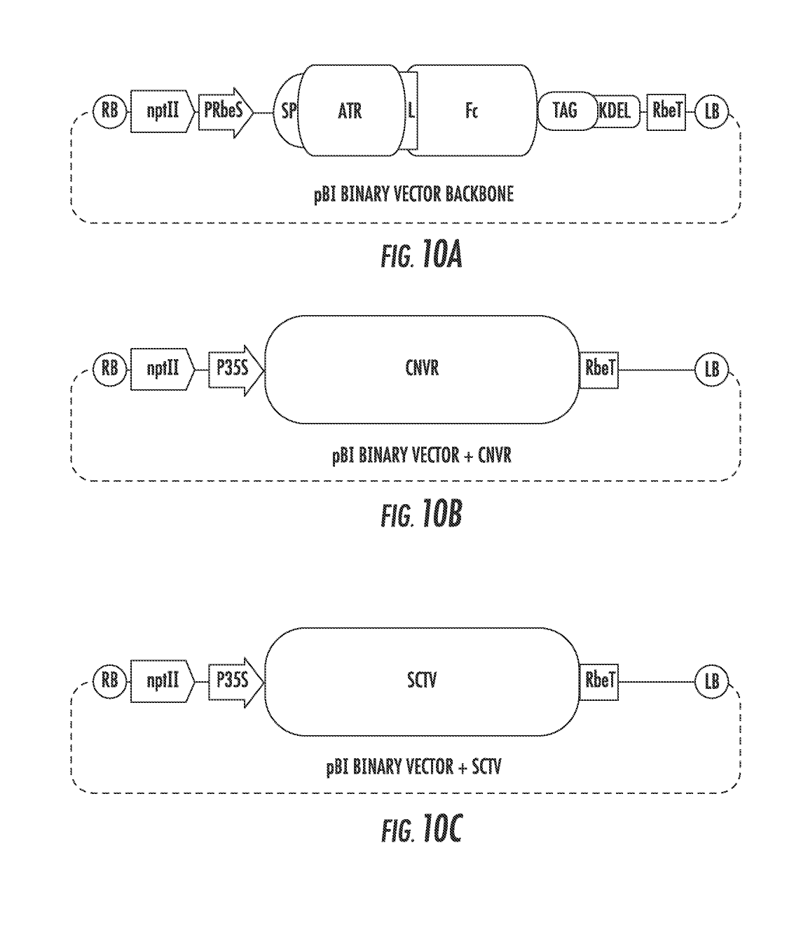

FIGS. 10A-10C illustrate schematic drawings of binary pBI-based vectors prepared for stable transformation of plants via Agrobacterium tumefaciens. The plasmid contains backbone of conventional pBI binary vector covalently linked to the T-DNA surrounded by the right border, RB, and the left border, LB. The T-DNA includes the nptII gene for kanamycin selection. The nptII gene is linked to the expression cassette that includes the Rubisco promoter, PrbcS or 35S promoter; the recombinant protein, the purification tag, Tag, the endoplasmic reticulum compartment sorting signal, KDEL, and the translation termination signal of the Rubisco gene, RbcT. FIG. 10A illustrates a vector for production of TBL-Fc protein. FIG. 10B illustrates a vector for cyanovirin (CNVR) microbicide generation. FIG. 10C illustrates a vector designed production of the scytovirin (SCTV) microbicide.

The simple and efficient Agrobacterium-mediated methods for transformation of Goldenseal, Echinacea, Kalanchoe, Thymus and Calendula were developed. These methods were used for initiating production of recombinant pharmaceutical proteins in medicinal plants. Due to the medicinal properties of goldenseal, Kalanchoe, Echinacea, Thyme and Calendula, recombinant pharmaceutical proteins, such as vaccines and microbicides, can be directly produced in these plants and used for treatment of the patients in needs thereof without purification. Additionally, unique medicinal properties of these plants might increase and supplement the efficacy of recombinant pharmaceuticals.

### Example 5

### Transformation of Callus Using Particle Bombardment

The plasmid pBI121 containing the nptII gene driven by the NOS promoter for kanamycin selection and the reporter GUS gene driven by the CaMV 35S promoter was used for the development of the transformation protocol for callus tissues and cells.

Callus cells of the medicinal plants were transformed using the PDS-1000/He system (Bio-Rad, CA, USA). Briefly, 6 mg of 1.0-micron gold particles were transferred to the sterile Treff Eppendorf tube (Biochemical Resources International Inc., MA). 1 ml of 70% ethanol was added into the tube, and vortexed for at least 1 min. Particles were pelleted by centrifugation at 14,000 rpm for 2 min, the supernatant was removed and 1 ml sterile distilled water was added. After centrifugation at 14,000 rpm for 2 min., the supernatant was removed. For coating, the following components were added to the Eppendorf tube containing gold particles: 20 μg plasmid DNA, 250 μl of 2.5 M CaCl₂, 50 μl of 0.1 M spermidine and 230 μl sterile distilled water. The mixture was incubated on ice for 10 min. with frequent, gentle vortexing before centrifugation at 10,000 rpm for 1 min. The supernatant was carefully removed and particles were washed with 600 μl of 100% ethanol. After removal of the supernatant, the particles were suspended in 72 μl of 100% ethanol and vortexed for 10 seconds. For each bombardment, 6 μl of the DNA-gold suspension were spread over a macro carrier disk and air-dried in a laminar flow hood for 5 min.

Two days before the bombardment, callus cells were placed at the center (3 cm in diameter) of a Petri plate (100 mm) containing 20 ml of the callus propagation MSC2 medium (Table 1). The cells were bombarded with DNA-coated gold particles discharged with different rupture disk pressures (900 and 1100 psi) from 9.0 and 12.0 cm distance between the stopping screen and the target tissue. Cells were bombarded one or two times per plate. After 3-4 days of cultivation on MSC2 medium in the dark, callus cells were transferred to the selection MSC2 medium supplemented with 30 mg/l or 50 mg/l kanamycin. Callus was transferred every 3-4 weeks to fresh selection medium. After 5-6 weeks of selection, kanamycin-resistant clones were tested by the histochemical GUS assay.

Histochemical determination of the expression of the 0-glucuronidase gene (GUS assay) was performed after 2, 20, 30 and 50 days after bombardment to optimize transformation protocol for Goldenseal, Echinacea, Kalanchoe, Calendula and Thyme callus cells (Jefferson et al. 1987 Plant Mol Biol Rep 5: 387). Briefly, callus cells were incubated overnight at 37° C. in solution containing 0.1 M NaPO₄ buffer, pH 7.0, 0.5 mM K-Ferricyanide, 0.01 M EDTA, 1 mg/ml X-gluc and 0.3% Triton X-100. GUS activity was recorded as blue staining using a light microscope.

### Example 6

### Production of the Recombinant Microbicides in Echinacea and Goldenseal

Nucleotide sequences of microbicide proteins were optimized for the expression in transgenic medicinal plants and synthesized using conventional techniques.

The HIV entry inhibitor cyanovirin, CNVR, was chosen for production in Echinacea and Goldenseal expression systems (Huskens D and Schols D, 2012, Algal Lectins as Potential HIV Microbicide Candidates, Marine Drugs: 10, 1476-1497). The Cyanovirin polypeptide PgCNVR-a designed for targeting into apoplast was as follows.

[SEQ ID NO: 1]
MSLSQNQAKFSKGFVVMIWVLFIACAITSTEASLGKFSQTCYNSAIQGSV
LTSTCERTNGGYNTSSIDLNSVIENVDGSLKWQPSNFIETCRNTQLAGSS
ELAAETRAQQFVSTKINLDDHIANIDGTLKYE

The nucleic acid sequence encoding the recombinant CNVR protein was optimized for plant expression as follows:

[SEQ ID NO: 2]
TCTAGATGTCCCTCTCACAGAACCAGGCTAAGTTCTCCAAGGGATTCGTG
GTGATGATCTGGGTGCTCTTTATCGCTTGCGCTATCACTTCCACTGAGGC
TTCTCTCGGAAAGTTCTCCCAGACTTGCTACAACTCCGCTATCCAGGGAT
CTGTGCTCACTTCTACTTGCGAGAGGACTAACGGTGGCTACAACACTTCC
TCCATCGATCTCAACTCCGTGATCGAGAATGTGGATGGCTCTCTTAAGTG
GCAGCCATCCAACTTCATCGAGACTTGCAGAAACACTCAGCTCGCTGGCT
CATCTGAACTTGCTGCTGAATGTAAGACTAGGGCTCAGCAGTTCGTGTCC
ACTAAGATCAACCTCGATGATCACATTGCTAACATCGATGGCACTCTCAA
GTACGAGTGAGAGCTC

A synthetic cDNA encoding for 11 kDa CNVR-a protein [SEQ ID NO:1] was cloned into a pBI121 derivative binary vector as shown in FIG. 10B. Referring to this figure, the transformation vector included the nptII gene for kanamycin selection of the transgenic plants. The CaMV 35S promoter was used to drive the expression of the transgenes in leaves. The Agrobacterium-mediated method described herein has been used in these experiments.

Another microbicide Scytovirin, SCTV, was also used for transformation experiments. The Scytovirin polypeptide, PgSCTV-a, designed for targeting into the apoplast was as follows:

[SEQ ID NO: 3]
MSLSQNQAKFSKGFVVMIWVLFIACAITSTEASGPTYCWNEANNPGGPNR
CSNNKQCDGARTCSSSGFCQGTSRKPDPGPKGPTYCWDEAKNPGGPNRCS
NSKQCDGARTCSSSGFCQGTAGHAAA.

The nucleic acid sequence encoding the recombinant SCTV protein was optimized for plant expression as follows:

[SEQ ID NO: 4]
TCTAGATGTCCCTCTCACAGAACCAGGCTAAGTTCTCCAAGGGATTCGTG
GTGATGATCTGGGTGCTCTTTATCGCTTGCGCTATTACTTCCACTGAGGC
TTCCGGACCTACTTACTGTTGGAACGAGGCTAACAATCCTGGTGGACCAA
ACAGGTGCTCCAACAACAAGCAATGTGATGGCGCTAGGACTTGCTCCTCT
TCAGGATTTTGTCAGGGCACTTCCCGTAAGCCAGATCCAGGACCAAAGGG
ACCAACTTATTGCTGGGATGAGGCAAAGAATCCAGGCGGTCCTAATAGGT
GCTCTAACTCCAAACAGTGTGATGGTGCTCGTACTTGCTCTAGTTCTGGA
TTCTGCCAAGGTACTGCTGGACATGCTGCTGCTTAAGAGCTC

A synthetic cDNA encoding the Scytovirin protein was cloned into pBI121-derived vector under 35S promoter. FIG. 10C shows a schematic drawings of a vector for production of the SCTV microbicide. Transgenic Echinacea plants were produced after transformation and selection with this construct.

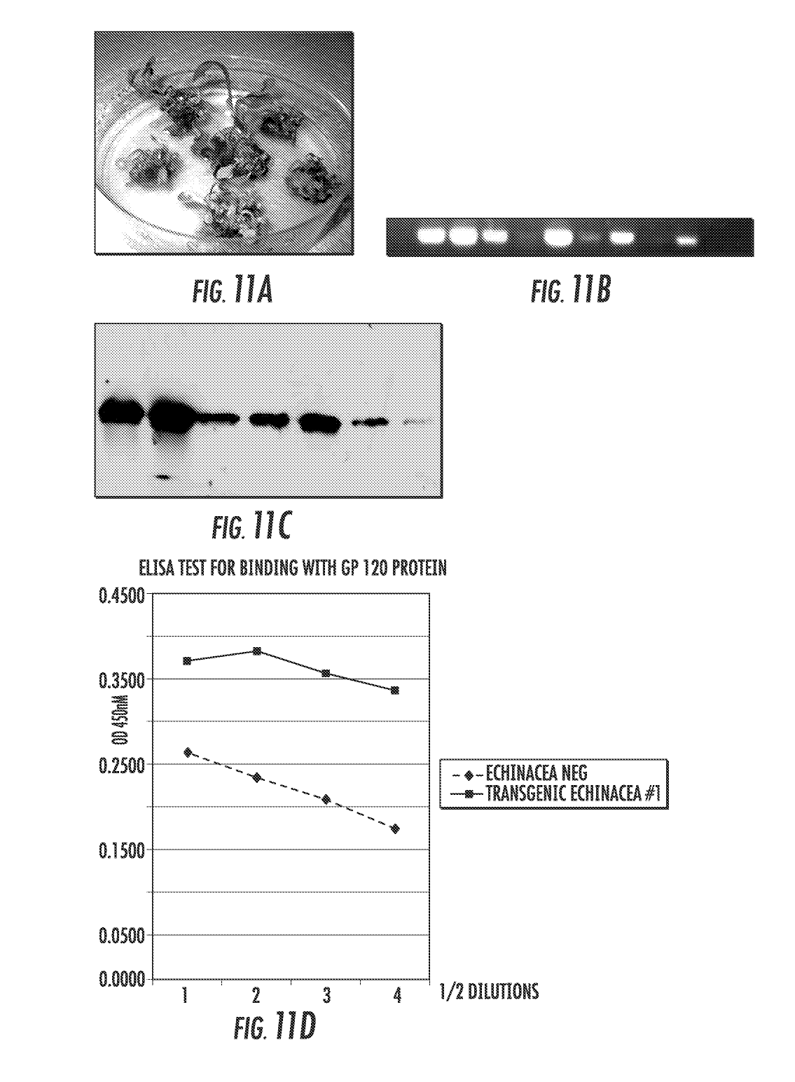

After transformation and selection on kanamycin-containing medium, the putative transgenic Echinacea and Goldenseal plants have been identified and tested by PCR, ELISA and Western blot. FIG. 11A illustrates selection of putative transgenic Echinacea shoots resistant to 50 mg/l of kanamycin. FIG. 11B illustrates target gene-specific PCR analysis of transgenic shoots. FIG. 11 C illustrates an example of the Western blot detection of the recombinant protein target using a polyclonal mouse primary antibody against the microbicide produced in E. coli. Binding of plant-derived microbicide to gp120 of HIV-1 was determined by the sandwich ELISA assay. Briefly, the Nunc Maxisorp ELISA plates were coated overnight at 4° C. with 100 μl of the gp120 protein (strain IIIB, Protein Sciences Corp., Meriden, Conn.) at 1 μg/ml in PBS, blocked for 1 h at room temperature with Blocking Buffer containing 3% BSA in PBS+0.05% Tween (PBS-T_{0.05}), and washed 3 times with PBS. Transgenic protein extracts 2 times diluted were added to PBS and incubated for 1 hour at room temperature. Extracts were washed 3× with PBS following incubation with primary polyclonal mouse antiserum (Antibodies Incorporated, Davis, Calif.) against the microbicide expressed in E. coli diluted 1:1000 in Blocking Buffer for 1 hour at room temperature, followed by three washing with PBS-T and incubation with the secondary goat anti-mouse secondary antibody HRP-conjugate (1:10,000 dilution in Blocking Buffer) for one hour. Finally, plates were washed 3 times with PBS-T and 1× with PBS. 100 μl of SureBlue TMB Microwell Peroxidase Substrate (KPL, EMD Millipore Corp., Temecula, Calif.) were added and developed in the dark for 10 minutes. The reaction was stopped by addition of 100 μl of 1N sulfuric acid (H₂SO₄). Absorbance was read at 450 nm using a BioTek Synergy HT plate reader. FIG. 11D illustrates that transgenic Echinacea plant expressed the recombinant CNVR protein that bind the HIV envelope protein gp120. The microbicides produced in Echinacea or Goldenseal may effectively inhibit HIV infection and can be used in the form of a minimally processed plant extract for anti-HIV preparations for direct vaginal application.

### Example 7

### Production of Anthrax-Binding Chimeric Recombinant Protein, TBL, Fusion with Fc in Echinacea and Kalanchoe

Echinacea and Kalanchoe have been transformed with the vector containing TBL anthrax toxin binding recombinant protein gene driven by Rubisco promoter. FIG. 10A schematically represents a plant expression vector. As shown, the vector includes a backbone derived from a conventional pBI binary vector covalently linked to the T-DNA surrounded by the right border, RB, and the left border, LB. The T-DNA includes the nptII gene for kanamycin selection. The nptII gene is linked to the expression cassette that includes the Rubisco promoter or CaM-35S promoter, PrbcS; the recombinant protein, the apoplast SP or endoplasmic reticulum compartment sorting signal, KDEL, and the translation termination signal of the Rubisco gene, RbcT. The PgA1B1 nucleic acid sequence encodes a variant of TBL polypeptide, the recombinant Anthrax Toxin Receptor polypeptide, fused with human IgG Fc, The PgA1B1 sequence optimized for expression in plants was as follows:

[SEQ ID NO: 5]
AGTCCCATGGAACAACCATCTTGCCGTAGGGCTTTCGATCTCTACTTCGT
GCTCGATAAGTCCGGCTCTGTTGCTAACAACTGGATCGAAATCTACAACT
TCGTGCAGCAGCTCGCTGAGAGATTCGTTTCTCCAGAGATGAGGCTCTCC
TTCATCGTGTTCTCTTCACAGGCTACTATCATCCTCCCACTCACTGGTGA
TAGGGGCAAGATTTCTAAGGGACTCGAGGATCTCAAGAGGGTGTCACCAG
TTGGAGAGACTTACATTCACGAGGGACTCAAGCTTGCTAACGAGCAGATT
CAAAAGGCTGGCGGCCTCAAGACTTCCTCCATTATTATCGCTCTCACTGA
TGGCAAGCTCGATGGACTTGTTCCATCCTACGCTGAGAAAGAGGCTAAGA
TCAGTCGTTCCCTTGGCGCTTCTGTTTACTGCGTTGGAGTGCTTGATTTC
GAGCAGGCTCAGCTTGAGAGGATCGCTGATTCCAAAGAGCAGGTTTTCCC
AGTTAAGGGCGGATTCCAAGCTCTCAAGGGCATCATCAACTCCATCCTTG
CTCAGTCTTGTACTGAGGGTGGTGGATCCGGAAACTCCGATAAGACTCAT
ACTTGTCCACCATGCCCAGCTCCAGAACTTCTTGGAGGACCATCTGTGTT
CTTGTTCCCACCAAAGCCAAAGGATACTCTCATGATCTCCAGGACTCCAG
AGGTTACATGCGTTGTGGTTGATGTGTCTCACGAGGATCCAGAGGTGAAG
TTCAACTGGTATGTGGATGGTGTTGAGGTGCACAACGCTAAGACTAAGCC
ACGTGAGGAACAGTACAACTCCACTTACAGGGTGGTGTCTGTGCTTACTG
TGCTTCACCAGGATTGGCTCAACGGCAAAGAGTACAAGTGCAAGGTGTCC
AACAAGGCTCTCCCAGCTCCAATCGAAAAGACTATCTCCAAGGCTAAGGG
ACAGCCAAGGGAACCACAGGTTTACACTCTTCCACCATCCAGGGAGAGAT
GACTAAGAACCAGGTGTCCCTTACTTGCCTCGTGAAGGGATTCTACCCAT
CCGATATTGCTGTTGAGTGGGAGTCTAATGGCCAGCCAGAGAACAACTAC
AAGACTACTCCACCAGTGCTCGATTCCGATGGCTCATTCTTCTTGTACTC
CAAGCTCACTGTGGATAAGTCCAGGTGGCAGCAGGGAAACGTTTTCTCTT
GCTCTGTTATGCACGAGGCTCTCCACAACCACTACACTCAGAAGTCCTTG
TCCTTGTCCCCAGGCAAGGATCTTATTGAGGGAAGAAGATCTCCAA

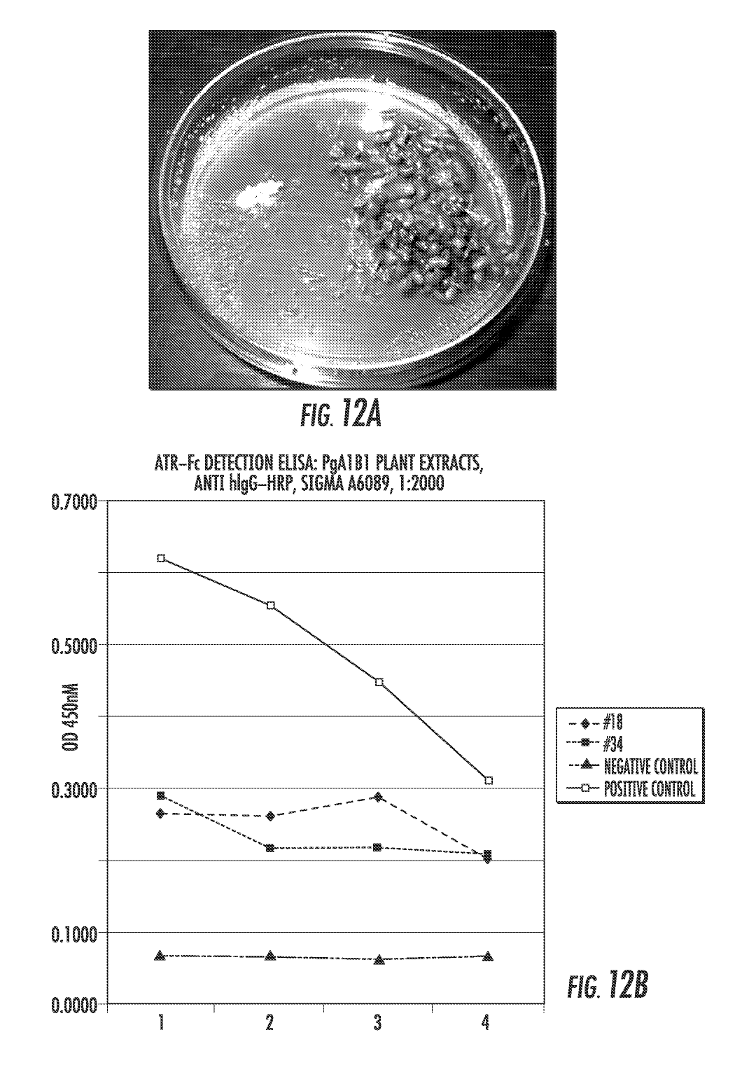

Agrobacterium strain LBA 4404 carrying the pBI binary vector for TBL-Fc protein expression was used for transformation experiments. Putative transgenic plants have been produced for Echinacea and Kalanchoe and tested for expression of recombinant protein. FIG. 12A illustrates the stringency of kanamycin selection. Referring to this figure, the transgenic shoots shown on the right side of the plate developed on the medium supplemented with kanamycin while the non-transgenic tissue shown on the left side of the plate died.

In vitro characterization and quantification of TBL-Fc protein expression was performed by ELISA. FIG. 12B illustrates a target-specific detection of the recombinant protein TBL in the extracts from the transgenic Kalanchoe plants by ELISA using the anti-human IgG peroxidase conjugate (Sigma, Cat. No. A-6089).

Extraction of soluble protein from transgenic Kalanchoe and Echinacea plants. Total and soluble plant proteins were extracted from transgenic Kalanchoe and Echinacea plants as described by Golovkin et al. 2007 Proc Natl Acad Sci USA 104: 6864. Plant tissue sample were collected, immediately frozen in liquid nitrogen and stored at −80° C. until extraction. Recombinant product was extracted from frozen plant tissues directly using equal amount (V/W) of Laemmli loading buffer for the total/insoluble extract or soluble buffer containing 0.1 M Na phosphate pH 7.4, 0.3 M NaCl, 3% Glycerol, 0.1 mM μ-ME and 0.05% of plant proteinase inhibitors cocktail (Sigma) for a total soluble protein, concentrated and brought into an equal volume of loading buffer.

### Example 8

### Transient Expression with Viral Vectors: Development of Plant-Based Vaccine Against Smallpox

A DNA fragment encoding vaccinia virus glycoprotein B5 membrane antigen was chosen for the transient production in planta. Goldenseal and Echinacea plants were used for transient transformation experiments.

A full extracellular antigenic domain (amino acids 20-275) of the vaccinia virus (VV) strain WR that contains major neutralization epitopes of the B5 glycoprotein (42 kDa) was initially selected for optimization. The B5 expression cassettes were designed to include C-terminal KDEL signals for ER targeting, c-Myc or His6 tags. Further optimization of the B5 extracellular antigenic domain that had no signal peptide transmembrane domain and cytoplasmic tail (Gene VACWR187) resulted in Pg1 constructs.

B5/Pg1 expression cassettes. The nucleic acid sequences encoding the B5 extracellular antigenic domain of EEV B5 Vaccinia virus glycoprotein (strain WR, GI:29692293) that has no signal peptide (amino acids 20-275), transmembrane domain and cytoplasmic tail, harbors three N-linked glycosylation sites located within the short consensus repeats (SCR2) and includes four modular SCR domains (amino acids 20-237) and the stalk region (amino acids 238-275) harboring sites of major neutralization epitopes was optimized for expression in plants. The plant optimized sequence was named pB5 (the plant optimized B5), or Pg1. The nucleic acid sequence encoding the Pg1 protein was as follows.

[SEQ ID NO: 6]
CCATGGCTTGAAACAAAAATGATTGTGCTTTCTGTGGGATCTGCTTCTTC
TAGTCCTATCGTGGTGGTTTTCTCTGTGGCATTACTCCTCTTCTATTTCT
CTGAAACATCTTTAGGTTGTACCGTTCCTACTATGAATAACGCTAAGTTG
ACTAGTACAGAGACCTCTTTTAATGATAAGCAAAAGGTTACTTTCACATG
TGATCAGGGATACCATTCTTCAGATCCTAATGCAGTGTGCGAGACTGATA
AGTGGAAATATGAAAACCCTTGTAAGAAAATGTGCACAGTTTCAGATTAC
ATCAGTGAGCTCTACAATAAGCCTCTCTATGAAGTGAACTCTACCATGAC
TCTTTCATGTAATGGTGAAACAAAGTACTTTAGATGCGAAGAAAAGAATG
GTAACACCTCATGGAATGATACAGTTACCTGTCCTAACGCTGAGTGCCAA
CCACTTCAGTTGGAACATGGTTCATGTCAACCAGTGAAGGAGAAGTACAG
TTTCGGAGAATACATGACAATTAATTGTGATGTTGGTTACGAAGTGATTG
GAGCTAGTTATATCTCTTGCACTGCAAATAGTTGGAACGTTATTCCTTCT
TGTCAACAGAAGTGCGATATGCCATCACTTAGTAATGGTTTGATCTCTGG
ATCAACATTTTCTATTGGTGGAGTTATCCACCTTTCATGCAAGAGTGGTT
TCACTTTGACAGGATCACCAAGTTCTACTTGTATTGATGGAAAGTGGAAT
CCTGTTCTTCCAATCTGCGTGAGGACCAACGAAGAGTTTGATCCTGTTGA
TAGATGGACCAGATGATGAGACTGATCTTTCTAGCTCTCAAAAGATGTTG
TGCAATACGAACAGGAGATTGAATCTTTGGAAGCAACTTATCATCACCAT
CACCACCACTCAAAAAGTTGGAATAGAGCACAGTTCGGTTCACATCATCA
TCATCATCACTAAAAGCTTAATTAAGAATTC

For transformation, Goldenseal and Echinacea were dipped into the bacterial suspension and vacuum pressure of 0.5-0.9 bar was applied for 2 minutes to facilitate Agrobacterium infiltration of leaf tissues. Plants were incubated in growth chamber for 7-10 days for maximum protein expression, harvested and analyzed.

“Magnifection” a transient production system was used for rapid expression of antigens in plants. Proteins were readily detected in the extracts of transfected leaves when expression was targeted to the apoplast area.

Electro-competent Agrobacterium cells were prepared in LB medium supplemented with 50 μg/ml rifampicin as overnight bacterial culture. The pelleted culture was washed twice with ice-cold sterile 10% glycerol and resuspended in 10 ml 10% glycerol to make 25 μl aliquots frozen in liquid nitrogen and stored at −80° C. For electroporation, 1 μl (0.1 μg) of plasmid DNA (Qiagen miniprep) was mixed with 25 μl electrocompetent Agrobacterium cells strain GV3101 in LB medium supplemented with 25-50 μg/ml Gentamycin, 10 μg/ml rifampicin and electroporated. Following electroporation, samples were incubated in 1 ml LB for at least 2-3 hours at 28° C. and at 120 rpm. The bacterial cells were plated onto selection LB media and incubated for 2-3 days at 28° C. Glycerol stocks for further use were prepared as 1:1 mix of 30% sterile glycerol with fresh overnight bacterial culture and stored at −80° C.

The “deconstructed” tobamovirus replicon magnICON system that was used for transformation was provided by Icon Genetics GmbH as described in Gleba et al. 2005 Vaccine 23: 2042, Marillonnet et al. 2005 Nat Biotechnol 23: 718; Marillonnet et al. 2004 Proc Natl Acad Sci USA 101: 6852, all of which are incorporated herein by reference as if fully set forth. Goldenseal and Echinacea plants were routinely transformed with the fresh overnight three-component cultures prepared from glycerol stocks in selective LB media incubated at 28° C. for 24 hours and mixed in equal proportions immediately prior the transformation experiments. One component (Agrobacterium cells) carried genes encoding the PG1 variants subcloned into the pICH11599 vector. The expression cassettes that included genes of interest were subcloned into the pICH11599 within the polylinker for NcoI-SacI, or NcoI-HindIII, or NcoI-EcoRI restriction sites. The other two components included the pre-manufactured vectors carrying either the targeting signal (cytosolic pICH10570) and the pICH10881 carrying the integrase as described in Giritch et al. 2006 Proc Natl Acad Sci USA 103(40): 1470, which is incorporated herein by reference as if fully set forth. The synthetic coding sequences were sub-cloned from the plasmid DNA supplied by synthesis facility Geneart (Life Technologies) and Genescript USA. The pICH11599 constructs were given the names of the corresponding sequences encoding antigenic proteins such as Pg1. All genes of interest included the ATG start codon within the 5′ NcoI site. An additional amino acid could be added at the N-terminal end of the expressed protein. In constructs designed for expression in cytosol (pICH10570), the protein was expressed from the first ATG of the cloned gene. All plasmids had a carbinicillin/ampicillin resistance gene for propagation in bacteria. Three vector-based components were mixed and diluted at least ten times with the Infiltration Buffer (IB) (10 mM MES-NaOH; pH 5.5; 10 mM MgSO₄).

The pB5 protein was readily detected at 6-8 days postinfection in the leaf tissues of Goldenseal and Echinacea transfected with the B5 and apoplast-targeting construct.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

What is claimed is:

1. A genetically engineered plant that belongs to the genus selected from the group consisting of: Hydrastis, Echinacea, Thymus, Calendula and Kalanchoe.

2. The genetically engineered plant of claim 1, wherein the plant is selected from the group consisting of: Hydrastis canadensis, Echinacea purpurea, Calendula officinalis, Thymus vulgaris, and Kalanchoe pinnata.

3. The genetically engineered plant of claim 1, wherein the plant includes a nucleic acid encoding a recombinant protein.

4. The genetically engineered plant of claim 3, wherein the recombinant protein is selected from the group consisting of: a microbicide, an antibody and an antigen.

5. The genetically engineered plant of claim 4, wherein the microbicide is a cyanovirin or a scytovirin.

6. The genetically engineered plant of claim 5, wherein the microbicide includes an amino acid sequence with at least 90% identity to a reference sequence of SEQ ID NO: 1 or SEQ ID NO: 3.

7. The genetically engineered plant of claim 4, wherein the antibody is an anthrax toxin binding recombinant antibody.

8. The genetically engineered plant of claim 4, wherein the antigen is a Vaccinia virus glycoprotein B5 membrane antigen.

9. The genetically engineered plant of claim 3, wherein the nucleic acid includes a sequence with at least 90% identity to a reference sequence selected from the group consisting of: SEQ ID NO: 2, SEQ ID NO: 4, SEQ ID NO: 5, and SEQ ID NO: 6.

10. The genetically engineered plant of claim 1, wherein the plant is stably transformed with a nucleic acid encoding the recombinant protein.

11. The genetically engineered plant of claim 1, wherein the plant transiently expresses the recombinant protein.

12. The genetically engineered plant of claim 1, wherein the plant is a mutant.

13. A method for genetically engineering a plant comprising:
contacting a plant with a vector comprising a nucleic acid encoding a recombinant protein, and
selecting a genetically engineered plant expressing the recombinant protein, wherein the plant belongs to the genus selected from the group consisting of: Hydrastis, Echinacea, Thymus, Calendula and Kalanchoe.

14. The method of claim 13, wherein the step of genetically engineering includes performing a transformation procedure selected from the group consisting of: biolistic transformation, Agrobacterium-mediated transformation, electroporation with a plasmid DNA, a DNA uptake, virus-mediated transformation, and protoplast transformation.

15. The method of claim 13, wherein the recombinant protein is selected from the group consisting of: a microbicide, an antibody and an antigen.

16. The method of claim 15, wherein the microbicide is a cyanovirin or a scytovirin.

17. The method of claim 16, wherein the microbicide includes an amino acid sequence with at least 90% identity to a reference sequence of SEQ ID NO: 1 or SEQ ID NO: 3.

18. The method of claim 15, wherein the antibody is an anthrax toxin binding recombinant antibody.

19. The method of claim 15, wherein the antigen is a Vaccinia virus glycoprotein B5 membrane antigen.

20. The method of claim 13, wherein the nucleic acid includes a sequence with at least 90% identity to a reference sequence selected from the group consisting of: SEQ ID NO: 2, SEQ ID NO: 4, SEQ ID NO: 5, and SEQ ID NO: 6.

21. A method for genetically engineering a plant comprising obtaining a mutant plant, wherein the plant belongs to the genus selected from the group consisting of: Hydrastis, Echinacea, Thymus, Calendula and Kalanchoe.

22. The method of claim 21, wherein the step of genetic engineering includes performing a procedure selected from the group consisting of: in vitro culture, chemical mutagenesis, radiation mutagenesis, T-DNA insertion, transposon-based-mutagenesis and TILLING.

23. A method for producing a recombinant protein in a plant comprising:
genetically engineering a plant to include a nucleic acid encoding a recombinant protein; and
culturing a genetically engineered plant under conditions effective for expression of the recombinant protein, wherein the plant belongs to the genus selected from the group consisting of: Hydrastis, Echinacea, Thymus, Calendula and Kalanchoe.

24. The method of claim 23 further comprising isolating and purifying the recombinant protein.

25. The method of claim 23, wherein the recombinant protein comprises a therapeutically effective protein.

26. The method of claim 25, wherein the therapeutically effective protein is selected from the group consisting of microbicides, vaccines, antigens, growth factors, transcription factors, antibodies and enzymes.

27. A method of treating a subject against a disease, the method comprising:
genetically engineering a plant to include a nucleic acid encoding a recombinant protein capable of preventing, curing or eliminating at least one symptom of the disease in the subject, wherein the plant belongs to the genus selected from the group consisting of: Hydrastis, Echinacea, Thymus, Calendula and Kalanchoe;
harvesting a genetically engineered plant expressing the recombinant protein;
performing the step (i) or (ii):
(i) preparing a first composition that includes the genetically engineered plant, or a part thereof;
(ii) isolating the recombinant protein and preparing a second composition that includes the isolated recombinant protein; and
administering the first composition or the second composition to the subject in need thereof.

28. The method of claim 27, wherein the disease is selected from the group consisting of: acquired immunodeficiency syndrome, Anthrax, and Smallpox.

29. The method of claim 27, wherein the recombinant protein is selected from the group consisting of: a microbicide, an antibody and an antigen.

30. The method of claim 29, wherein the microbicide is a cyanovirin or a scytovirin.

31. The method of claim 29, wherein the microbicide includes an amino acid sequence with at least 90% identity to a reference sequence of SEQ ID NO: 1 or SEQ ID NO: 3.

32. The method of claim 29, wherein the antibody is an anthrax toxin binding recombinant antibody.

33. The method of claim 32, wherein the antigen is a Vaccinia virus glycoprotein B5 membrane antigen.

34. The method of claim 27, wherein the nucleic acid includes a sequence with at least 90% identity to a reference sequence selected from the group consisting of: SEQ ID NO: 2, SEQ ID NO: 4, SEQ ID NO: 5, and SEQ ID NO: 6.

35. The method of claim 27, wherein the subject is a mammal, or a human.

36. The method of claim 27, wherein the step of administering includes a route selected from the group consisting of: intravenous, intramuscular, intraperitoneal, intradermal, mucosal, cutaneous and subcutaneous administering.

37. The method of claim 36, wherein the step of administering includes contacting a mucosal surface of the subject.

38. The method of claim 37, wherein the mucosal surface is selected from the group consisting of: oral, lingual, sublingual, ocular, nasal, vaginal, urethral, and rectal surfaces.

39. The method of claim 27, wherein the first composition or the second composition is administered in an amount sufficient to prevent, cure or eliminate at least one symptom of the disease in the subject.

40. A method of propagating a plant in vitro comprising culturing a plant, or part thereof, on a culture medium that includes at least one plant growth regulator, and recovering multiple shoots from the plant, or part thereof, wherein the plant belongs to the genus selected from the group consisting of: Hydrastis, Echinacea, Thymus, Calendula and Kalanchoe.

41. The method of claim 40, wherein the at least one plant growth regulator is a cytokinin.

42. The method of claim 41, wherein the cytokinin is selected from the group consisting of: kinetin, benzylaminopurine, zeatin, and thidiazuron.

43. The method of claim 40, wherein the at least one plant regulator is an auxin.

44. The method of claim 43, wherein the auxin is selected from the group consisting of: indole-butyric acid, indole-acetic acid, naphthalene-acetic acid, and 2,4-dichlorophenoxy-acetic acid.

45. The method of claim 40 further comprising rooting the multiple shoots.

46. A method for producing a cell suspension culture comprising culturing a plant, part, or tissue thereof, in a liquid culture medium that includes at least one auxin, wherein the plant belongs to the genus selected from the group consisting of: Hydrastis, Echinacea, Thymus, Calendula and Kalanchoe.

47. The method of claim 46, wherein the at least one auxin is selected from the group consisting of: indole-butyric acid, indole-acetic acid, naphthalene-acetic acid, and 2,4-dichlorophenoxy-acetic acid.


SEQUENCE LISTING (6 sequences)

SEQ ID NO: 1 - protein, 132 aa, Artificial Sequence
  Synthetic construct, PgCNVR-a
MSLSQNQAKFSKGFVVMIWVLFIACAITSTEASLGKFSQTCYNSAIQGSVLTSTCERTNGGYNTSSIDLNSVIENVDGSLKWQPSNFIETCRNTQLAGSSELAAETRAQQFVSTKINLDDHIANIDGTLKYE

SEQ ID NO: 2 - DNA, 416 bp, Artificial Sequence
  Synthetic construct, polynucleotide encoding PgCNVR
tctagatgtccctctcacagaaccaggctaagttctccaagggattcgtggtgatgatctgggtgctctttatcgcttgcgctatcacttccactgaggcttctctcggaaagttctcccagacttgctacaactccgctatccagggatctgtgctcacttctacttgcgagaggactaacggtggctacaacacttcctccatcgatctcaactccgtgatcgagaatgtggatggctctcttaagtggcagccatccaacttcatcgagacttgcagaaacactcagctcgctggctcatctgaacttgctgctgaatgtaagactagggctcagcagttcgtgtccactaagatcaacctcgatgatcacattgctaacatcgatggcactctcaagtacgagtgagagctc

SEQ ID NO: 3 - protein, 126 aa, Artificial Sequence
  Synthetic construct, PgSCTV-a
MSLSQNQAKFSKGFVVMIWVLFIACAITSTEASGPTYCWNEANNPGGPNRCSNNKQCDGARTCSSSGFCQGTSRKPDPGPKGPTYCWDEAKNPGGPNRCSNSKQCDGARTCSSSGFCQGTAGHAAA

SEQ ID NO: 4 - DNA, 392 bp, Artificial Sequence
  Syntehtic construct, polynucleotide encoding PgSCTV
tctagatgtccctctcacagaaccaggctaagttctccaagggattcgtggtgatgatctgggtgctctttatcgcttgcgctattacttccactgaggcttccggacctacttactgttggaacgaggctaacaatcctggtggaccaaacaggtgctccaacaacaagcaatgtgatggcgctaggacttgctcctcttcaggattttgtcagggcacttcccgtaagccagatccaggaccaaagggaccaacttattgctgggatgaggcaaagaatccaggcggtcctaataggtgctctaactccaaacagtgtgatggtgctcgtacttgctctagttctggattctgccaaggtactgctggacatgctgctgcttaagagctc

SEQ ID NO: 5 - DNA, 1296 bp, Artificial Sequence
  Synthetic construct, PgA1B1
agtcccatggaacaaccatcttgccgtagggctttcgatctctacttcgtgctcgataagtccggctctgttgctaacaactggatcgaaatctacaacttcgtgcagcagctcgctgagagattcgtttctccagagatgaggctctccttcatcgtgttctcttcacaggctactatcatcctcccactcactggtgataggggcaagatttctaagggactcgaggatctcaagagggtgtcaccagttggagagacttacattcacgagggactcaagcttgctaacgagcagattcaaaaggctggcggcctcaagacttcctccattattatcgctctcactgatggcaagctcgatggacttgttccatcctacgctgagaaagaggctaagatcagtcgttcccttggcgcttctgtttactgcgttggagtgcttgatttcgagcaggctcagcttgagaggatcgctgattccaaagagcaggttttcccagttaagggcggattccaagctctcaagggcatcatcaactccatccttgctcagtcttgtactgagggtggtggatccggaaactccgataagactcatacttgtccaccatgcccagctccagaacttcttggaggaccatctgtgttcttgttcccaccaaagccaaaggatactctcatgatctccaggactccagaggttacatgcgttgtggttgatgtgtctcacgaggatccagaggtgaagttcaactggtatgtggatggtgttgaggtgcacaacgctaagactaagccacgtgaggaacagtacaactccacttacagggtggtgtctgtgcttactgtgcttcaccaggattggctcaacggcaaagagtacaagtgcaaggtgtccaacaaggctctcccagctccaatcgaaaagactatctccaaggctaagggacagccaagggaaccacaggtttacactcttccaccatccagggagagatgactaagaaccaggtgtcccttacttgcctcgtgaagggattctacccatccgatattgctgttgagtgggagtctaatggccagccagagaacaactacaagactactccaccagtgctcgattccgatggctcattcttcttgtactccaagctcactgtggataagtccaggtggcagcagggaaacgttttctcttgctctgttatgcacgaggctctccacaaccactacactcagaagtccttgtccttgtccccaggcaaggatcttattgagggaagaagatctccaa

SEQ ID NO: 6 - DNA, 981 bp, Artificial Sequence
  Synthetic construct, Pg1
ccatggcttgaaacaaaaatgattgtgctttctgtgggatctgcttcttctagtcctatcgtggtggttttctctgtggcattactcctcttctatttctctgaaacatctttaggttgtaccgttcctactatgaataacgctaagttgactagtacagagacctcttttaatgataagcaaaaggttactttcacatgtgatcagggataccattcttcagatcctaatgcagtgtgcgagactgataagtggaaatatgaaaacccttgtaagaaaatgtgcacagtttcagattacatcagtgagctctacaataagcctctctatgaagtgaactctaccatgactctttcatgtaatggtgaaacaaagtactttagatgcgaagaaaagaatggtaacacctcatggaatgatacagttacctgtcctaacgctgagtgccaaccacttcagttggaacatggttcatgtcaaccagtgaaggagaagtacagtttcggagaatacatgacaattaattgtgatgttggttacgaagtgattggagctagttatatctcttgcactgcaaatagttggaacgttattccttcttgtcaacagaagtgcgatatgccatcacttagtaatggtttgatctctggatcaacattttctattggtggagttatccacctttcatgcaagagtggtttcactttgacaggatcaccaagttctacttgtattgatggaaagtggaatcctgttcttccaatctgcgtgaggaccaacgaagagtttgatcctgttgatgatggaccagatgatgagactgatctttctaagctctcaaaagatgttgtgcaatacgaacaggagattgaatctttggaagcaacttatcatcaccatcaccaccactcaaaaagttggaatagagcacagttcggttcacatcatcatcatcatcactaaaagcttaattaagaattc